(19) United States
(12) Patent Application Publication
(10) Pub. No.: US 2005/0229265 A1
(43) Pub. Date: Oct. 13, 2005

(54) Insect ammunition vectors and methods of use to identify pesticide targets

(76) Inventors: Jonathan Scott Margolis, SAN FRANCISCO, CA (US); Margaret L. Winberg, San Francisco, CA (US); Stephen Thomas Thibault, San Mateo, CA (US); Allen James Ebens Jr, San Carlos, CA (US); Wesley Y. Miyazaki, San Francisco, CA (US); Casey Kopczynski, Chapel Hill, NC (US)

Correspondence Address: PATENT DEPT;EXELIXIS, INC., 170 HARBOR WAY, P.O. BOX 511, SOUTH SAN FRANCISCO, CA, 94083-0511, US

(21) Appl. No.: 10/474,881

(22) PCT Filed: Apr. 10, 2002
(86) PCT No.: PCT/US02/11383

§ 371 Date: Feb. 28, 2005

Related U.S. Application Data
(62) Division of application No. 60/282,950, filed on Apr. 10, 2001.

(51) Int. Cl. A01K 67/033; C12Q 1/00

(52) U.S. Cl. 800/13; 435/4

(57) ABSTRACT

The present invention provides methods for identifying pesticide targets and pesticidal agents using transposable elements in insects and insect cells lines. The invention provides engineered transposable elements for use in identification of pesticide targets. The invention further provides a biological array, a collection of transgenic insect lines or insect cell lines, the genome of each containing at least one transposable element that mutates one of the insect's genes, such that the complete collection contains a mutation in essentially every gene in the insect's genome.

### BACKGROUND OF THE INVENTION

In the field of agricultural biotechnology, there is a need for new pesticide targets, and for new biology-based methods for the development of efficacious compounds. The industry's traditional chemistry-based approach, which generally uses whole organism screening methods, is associated with numerous obstacles. This process is relatively slow, labor intensive, expensive and is unable to access the diversity of combinatorial chemical libraries due to the mass of chemical needed for each assay. Additionally, this approach generates compounds which deliver the desired effect, namely death or disablement of the insect pest, but for which the mechanism of action is often unknown. Since defining the protein target of a new pesticide is critical to meet licensing and regulatory requirements, this approach requires substantial investment of research into the biochemical and physiological effects of the compound on the organism. Furthermore, since the industry has often focussed on modifying existing compounds, several companies have one or multiple compounds with the same mode of action. Currently, two thirds of all pesticides currently sold act on only one of two molecular targets (namely, voltage gated sodium channels and acetylcholinesterases). The phenomenon of “overworked targets” may result in a high selection pressure on the target and eventually to cross-resistance to each of the relevant classes of pesticides. The outlawing of existing classes of pesticides such as the carbamates and organophosphates further highlights the need for and unique economic opportunities associated with developing pesticides with novel modes of action.

Biology-based approaches, which aim to first discover appropriate gene targets, and then use the protein products from these target genes to develop new pesticidal molecules, offer several advantages over chemistry-based approaches. Developing pesticides with known mechanisms of action facilitates the production of new compounds that are safer, selective, and more efficient. Potential lead compounds can be directly counter-screened on the same target cloned from human or beneficial insect sources to exclude broad-spectrum toxins. Target-based strategies use the techniques of high throughput screening developed in the pharmaceutical industry to test between 10⁵ and 10⁶ compounds per week for activity on the target. High-throughput assays can be run rapidly and inexpensively and, due to their scale, allow access to the structural variety granted by combinatorial chemistry. Knowing the target permits chemical analogs of an active compound to be rapidly tested in an in vitro assay to select for more effective and potent toxins. In addition, the molecular diversity inherent in the specific structures of targets may be exploited via combinatorial chemistry and high-throughput screening.

Transposable elements are naturally mobile pieces of DNA that can disrupt gene function when they insert into key sequences. As mutagens, transposable elements have the significant advantage of providing a molecular tag for easy identification of the disrupted gene (Bingham P M et al., Cell (1981) 253:693-704; Lai C, Genome (1994) 37:519-25). Furthermore, since they can be engineered to carry non-element DNA, they can carry sequences that act on genomic site to produce a number of effects, such as ectopic expression and chromosomal rearrangements. Sophisticated transposable element technologies exist for the model insect Drosophila melanogaster, and are being developed for numerous pest species (e.g., O'Brochta D A and Atkinson P W, Insect Biochem Mol Biol (1996) 26:739-753). In one exemplary application of transposon techniques to pest control, the use of engineered transposable elements to facilitate sterile insect technique has been proposed (Tibault S T et al., Insect Molecular Biology (1999) 8:119-123).

The present invention uses transposable elements as a tool to facilitate the development of new insecticides, thus synergizing advances in the biology-based and chemistry-based pest control strategies.

### SUMMARY OF THE INVENTION

The invention provides methods for identification of pesticides utilizing transposable element insertions in various insects and cultured insect cells. Transposable element insertions that are lethal to the insect or cultured insect cells are identified, leading to the identification of genes harboring the lethal insertions. Protein products of these lethal genes, or their orthologues, are then used to screen for agents that specifically inhibit the protein products. The inhibiting agents are identified as pesticides.

The methods of the invention are amenable to large scale screens, leading to the identification of a large number of pesticidal agents. The insect of the invention may be Drosophila or a crop pest species. Likewise, the cultured cells of the invention may be derived from Drosophila, or a crop pest species.

Preferred lethal genes of the invention are provided that encode enzymes, soluble proteins, and membrane proteins. Various screening assays for identification of candidate pesticides are also provided.

Various engineered transposable elements, including the XP element, are also provided. In one embodiment of the invention, the transposable element comprises splice trap sequences.

The invention further provides a biological array, a collection of transgenic insect lines or insect cell lines, the genome of each containing at least one transposable element that mutates one of the insect's genes, such that the complete collection contains a mutation in essentially every gene in the insect's genome.

### BRIEF DESCRIPTION OF THE FIGURES

FIG. 1 depicts the genetic crosses used to conduct a screen for pesticide targets in Drosophila, as further described in Example 1.

FIG. 2 depicts the genetic crosses to conduct a screen for pesticide targets in Heliothis virenscens, as further described in Example 2.

FIG. 3 depicts results of analysis that indicated that a piggyBac transposon comprising splice-trap sequences effectively generated lethal insertions when it inserted into intronic sequences in the Drosophila genome, as further described in Example 3.

### DETAILED DESCRIPTION OF THE INVENTION

The present provides novel methods for uncovering pesticide targets by systematic disruption of an insect's genes using transposable element insertions. The phenotype that results from a disrupted gene serves as a surrogate for chemical inhibition of the associated protein. Mutations that are lethal to the animal when homozygous identify genes that are critical for cell function and the animal's survival. These genes are often validated insecticide targets, and compounds that antagonize the gene products may be effective insecticidal agents.

In a preferred embodiment, the invention is useful for identifying potential targets for which function is known or may be assumed based upon homology to known genes or proteins. It therefore complements current genome sequencing and annotation projects, which can predict genes based on genomic sequence and assign putative function based on homology to known genes or proteins. In the absence of experimental data, however, such information is insufficient for predicting an organism's requirements for genes. The data provided by the methods of the invention are especially valuable for genes that belong to a family that has many members within one organism. For instance, the Drosophila genome is predicted to encode twenty nuclear hormone receptors (Adams M et al., Science (2000) 287: 2185-2195). Without the lethal insertion data, it would be near impossible to predict which ones are essential for viability.

In one aspect, the present invention comprises a large-scale effort to severally disrupt an insect's genes, either in the intact animal or in cultured cells, using transposable element-based insertional mutagenesis in order to identify proteins that are essential for viability and therefore candidate pesticide targets. The invention includes methods for mobilizing transposable elements in the host genome via expression of the corresponding transposase and identifying the novel transposable element insertions (the “insertions”). The invention further includes methods for determining which new insertions are lethal to the animal when homozygous, identifying the disrupted genes, and assessing their suitability as pesticide targets. As used herein, “pesticide” refers to chemical or biological agents that kill, paralyze, sterilize or otherwise disable pest species. Accordingly “lethal insertions,” as used herein, encompass insertions that kill, paralyze, sterilize, or otherwise disable the insect host. A “lethal gene,” as used herein, refers to a gene whose inactivation kills, paralyzes, sterilizes, or otherwise disables the host. The invention further includes methods for generating dominantly acting gene disruptions in lethal genes in cultured insect cells that serve as a surrogate for homozygous disruptions of lethal genes in the intact animal.

Any insect host that is amenable to genetic manipulation, can serve as a host for transposable elements, and is easily maintained in laboratory stocks may be used. Exemplary hosts are model systems, such as Drosophila melanogaster, which is hereinafter referred to as Drosophila (other Drosophila species are referred to by species designation), for which ample molecular data, genetic data, and/or genetic tools exist. These may not be the intended targets of the pesticides but are similar enough to actual pests that the data will be agriculturally relevant. For convenience, hosts for which these information and tools do not exist are referred to as “non-model hosts.” However, it is understood that directed research efforts can develop particular species into model systems. We have found that Drosophila is sensitive to many of the commercially successful insecticides, suggesting that targets defined in this species would be applicable to the control of a broad range of invertebrate pest species. Other preferred hosts are pest species that harm agricultural crops, act as parasites or disease vectors, damage structures, homes, or gardens, or cause hygienic or aesthetic damage. These include Lepidoptera, such as Plodia interpunctella, Pectinophora gossypiella, Manduca sexta, and Heliothis species, Coleoptera, such as Leptinotarsa decemlineata and Tribolium species, Hemiptera, such as Myzus species, and Diptera, such as Anopheles species. Additionally, the cultured cells of these model or pest insects may be used. As used herein, in vivo and in vitro refer, respectively, to whole organism and cultured cell methods. The “host” refers to the animal or cell that harbors or will harbor the ammunition vector in at least one copy or a representative animal or cell that is essentially genetically equivalent to the animal or cell that harbors or will harbor the ammunition vector. The “host system” refers to the animal or cultured cell system in which the lethality of the transposable element insertion is assessed.

Vector Design and Construction

As used herein the “ammunition vector” refers to the mobilized transposable element, and any accompanying sequences that are inserted into the host genome in order to disrupt a host gene. For in vivo application, the ammunition vector minimally contains the portions of the transposable element sequences required for transposition, and generally contains a marker gene. For in vitro application, the ammunition vector minimally contains the portions necessary for mobilization and generally contains sequences that will direct ectopic transcription of the flanking DNA, as well as a marker gene.

The ammunition vector may additionally contain different types of nucleic acid sequences to be inserted in the host organism. This may include any part of a gene, such as regulatory sequence encoded in upstream enhancers or promoters, 5′ or 3′ untranslated regions, or introns, or coding sequence, either full-length genes or gene fragments. Generally, non-coding sequences will act on coding sequences either within the ammunition vector or in the host organism's genome, or will act on or respond to another transgenic element. Coding sequence included within the vector may encode reporters, protein tags, or selection systems. Descriptions of sequences that can be included in the vector and their various utilities are provided below.

Transposable Element Component

The choice of transposable element will affect the nature of the insertions. Class II transposable elements, which are interchangeably referred to as “transposons”, are especially useful; since they transpose without an RNA intermediate, they can be used to carry exogenous DNA fragments (reviewed in O'Brochta D A and Atkinson P W, Insect Biochem Molec Biol (1996) 26:739-753). The ammunition vector typically derives from a Class II transposable element that has been engineered to retain the terminal sequences required for transposition, which are also referred to as the “transposon ends,” but lack the transposase. Any transposable element that can carry non-element DNA fragments, can be mobilized by a transposase source in trans, and can insert in the host of choice may be used for engineering the ammunition vector. The most efficient transposable elements insert in single copies into the host genome and mobilize at a high frequency when exposed to the mobilizing enzyme, preferably 1 in 1000 animals, more preferably 1 in 100, and most preferably 1 in 10.

Some transposable elements have chromosomal insertion site preferences. For instance, mobilization of the P element in Drosophila is associated with both “hotspots” and “cold spots,” respectively, regions of especially frequent and especially infrequent insertion (Spradling A C et al., Genetics (1999) 153:135-177). Use of a transposable element that has insertions site biases requires that the researcher generates and analyze a much larger collection of single insertions in order to achieve significant genomic coverage than is necessary if an element without these biases is used. In accordance with the present invention, it is preferable to use a transposable element that exhibits minimal insertion site bias such that it is possible to generate a collection of insertions that collectively represent genome-wide or near genome-wide coverage.

A key aspect of the system is the ability of the researcher to control transposition of the element, specifically, both to promote mobilization by supplying transposase and to prevent it by removing the source of transposase. As used herein, “transposition” refers both to the initial insertion into the host genome, or “transformation,” and to subsequent excisions and insertions, or “mobilization.” Individual transposons may be mobilized by the native transposase (i.e., the transposase associated with the naturally-occurring element) or, in certain circumstances, by another transposon's transposase via “cross-mobilization (O'Brochta D A and Atkinson P W, supra).” Although transposable elements that have been engineered as tools generally lack a cis-acting source of transposase, they are nonetheless susceptible to mobilization if the host organism harbors transposons that produce the native transposase or another transposase capable of cross-mobilization. Since this situation lessens the researcher's ability to control the element's activity, for any given host organism, the most preferable transposon ends will not be mobilized by any host-encoded transposase. Therefore, the most preferable species-specific transposons are those for which some existing strains lack the corresponding transposase. The most preferable promiscuous (i.e., active in multiple species) transposons are those that originate from a single carrier species and are able to transform a broad range of species only when the corresponding transposase is supplied. Examples of both kinds of elements are provided below.

In a preferred embodiment of the invention, an engineered piggyBac element from Trichoplusia ni (cabbage looper) is used for insertional mutagenesis in Drosophila or in pest insects. Sequence of the wild-type piggyBac transposon is provided in SEQ ID NO:1. piggyBac can transpose in a broad range of evolutionarily diverse insect species, including Diptera and Lepidoptera (Toshiki et al., Nature Biotechnology (2000) 18:81-84; Handler A M and Harrell R A II, Insect Molecular Biology (1999) 8:449-457; Peloquin J J et al., Insect Molecular Biology (2000) 9:323-333). Strict insertion site specificity and precise excisions distinguish the piggyBac element. piggyBac inserts into the tetranucleotide TTAA and restores the target site upon excision. As further discussed below, this allows the researcher to excise a mutagenic element and expect reversion of any transposon-induced phenotype. We have developed a piggyBac gene tagging system and generated a large-scale collection of Drosophila lines containing genomic piggyBac insertions. Molecular and genetic analyses of the collection, as presented in the Examples, have permitted a detailed analysis of piggyBac's insertion and mobilization characteristics and have elucidated the utility of piggyBac for identifying pesticide targets. In some applications, the entire approximately 350 base pair (bp) transposon ends may be used; the piggyBac 5′ end corresponds to nucleotides (nt) 1-332 of SEQ ID NO: 1, and the piggyBac 3′ end corresponds to nt 2126-2480 of SEQ ID NO: 1. Alternatively, approximately 100 bp 5′ and 3′ transposon ends, which correspond to, respectively, nt 1-105 of SEQ ID NO: 1 and nt 2375-2480 of SEQ ID NO: 1, and which we have shown mobilize as efficiently as the full-length ends in Drosophila embryos, are used. It can be readily determined if shorter portions of the piggyBac transposon ends are also effective in achieving transposition, and if so, they may be used in the present invention.

If the host organism is Drosophila, another preferred transposon is the P-element (reviewed in Ashburner, Drosophila: A Laboratory Handbook, Cold Spring Harbor Laboratory Press, 1989, pp.1017-1063). The P-element only transposes in a subset of Drosophila species and its primary usage as a genetic tool has been in the original carrier Drosophila melanogaster. Many laboratory Drosophila strains lack endogenous P-elements and P-transposase. When exogenously introduced, P-elements insert at relatively high frequency, typically ˜5-20% and most often yield single insertions. P-elements have shown a strong bias for the 5′ ends of genes (Spradling et al., PNAS (1995) 92:10824-30).

Although P elements are biased to certain chromosomal loci and to the 5′ regions of genes, they are still extremely useful due to their widespread use as genetic tools, the large existing collection of P-elements, and the wealth of data about them (Spradling et al., 1999). For instance, many P element hotspots have been well characterized, facilitating, for example, methods to rapidly eliminate such insertions into hotspots from a collection (Spradling et al., 1999). Moreover, as described below, we have generated an optimized P (XP) element for generation of lethal mutations.

Members of the hAT family of plant and insect transposons are alternative preferred elements. The hobo member element originates from and has been used as a gene vector in Drosophila. Compared to the P-element, the hobo element has similar transposition rates, but may display different insertion site biases (Smith D et al., Genetics (1993) 135:1063-1076). The Hermes element, from the housefly Musca domestica may be used in Drosophila and other pest species (Warren W D, et al., Genetical Research (1994) 64:87-97). Hermes, which is closely related to hobo, can carry non-element DNA and displays a 35% transposition rate in Drosophila melanogaster (O'Brochta D A et al., Genetics (1996) 142:907-14). Both hobo and Hermes transpose in a broad range of dipteran and leptidopteran insects (reviewed in O'Brochta DA and Atkinson P W. Insect Biochemistry and Molecular Biology (1996) 26:739-53). Another hAT element, Tag1 from Arabidopsis thaliana, is under investigation for use in insects.

Other preferred elements for transposition in multiple species belong to the mariner superfamily (reviewed in Plasterk R H, Trends in Genetics (1999) 15:326-32). Transposons from this family may be the most widespread DNA transposon in nature and have been found in fungi, plants, ciliates, and many animal species. These elements' ability to transpose in a broad range of invertebrate species and cultured cells makes them especially attractive for transposition in insect and other species that do not already have well-characterized genetic tools. The mariner element, isolated from Drosophila mauritania, has been used for germline transformation in Drosophila melanogaster and Aedes aegypti and can carry inserts up to approximately 2.2 kb. Sleeping Beauty, Himar1, Minos, Tc1 and Tc3 are other preferred members of the mariner family that transpose in diverse species.

Marker Gene

In addition to the regulatory sequences, the transposable element usually contains a marker for transposition. The marker is desired for in vivo use, for it indicates initial insertion into the host genome, but may be dispensable for in vitro uses. In the animal, the marker further indicates segregation of genetic or transgenic elements and, if it is dose-dependent, homozygosity of the transposable element The transposition marker is hereinafter also referred to as the “primary marker”. Any gene can be used as a marker that causes a reliable and easily scored phenotypic change in transgenic animals and that does not interfere with the screening methods. In Drosophila, for instance, preferred marker genes include white and rosy which affect eye color (a comprehensive list of markers may be found in Ashburner, supra, pp. 299-418). Other preferred markers are non-endogenous genes. A gene for drug resistance allows selection or rescue specifically of transformed individuals or cells (e.g., Pfeifer T A et al, Gene (1997) 188:183-190). Luciferase and β-Galactosidase (lacZ) are luminescent and chromogenic markers, respectively, that are useful in a variety of fixed tissues and cells (Gould S J and Subramani S, Analytical Biochemistry (1988) 175:5-13; O'Kane C J and Gehring W J, PNAS (1987) 84:9123-9127). Fluorescent proteins, such as green fluorescent protein (GFP) and its derivatives from the jellyfish A. victoria or fluourescent proteins from sea coral, are especially useful (Chalfie et al., Science (1994) 263:802-805; Miller D M 3rd et al., Biotechniques (1999) 26:914-918, 920-921; Matz M V et al, Nat Biotechnol (1999) 17:969-973.). They appear universally active and are detected in the live animal or cell. For eye-bearing animals, a universal marker of transgenesis that includes a fluorescent marker gene, driven by an artificial promoter that is active in eye tissue of most arthropods, exemplified by the system described in Berghammer A J et al. (Nature (1999) 402:370), may be used.

Genetic and Molecular Tools

In addition to the marker gene, the ammunition vector may contain additional DNA sequences that facilitate genetic or molecular analysis of the disrupted gene, or that contribute to gene disruption. As further described below, an initial isolation of a putative lethal gene will generally be followed by subsequent experimentation that further characterizes its suitability as a pesticide target. Sometimes, when assessing lethality of insertions is especially time-consuming for a given host, the “supplemental” analysis of the disrupted gene, such as expression analysis, will accompany or precede the assessment of lethality. A screen that uses expression analysis to help predict the lethal insertions is provided in the examples.

Preferred sequences facilitate expression analysis of the disrupted genes. The “enhancer trap” is an in vivo method for characterizing the expression pattern from a novel gene; it has been widely use in Drosophila and may be adapted to use in other insects (O'Kane C J and Gehring W J, PNAS (1987) 84:9123-9127; Mollereau B et al., Mech Dev (2000) 93:151-60). The ammunition vector contains a reporter gene, such as lacZ or GFP, under a minimal promoter. The construct is sensitive to the regulatory elements of the region where it inserts. As a result, detection of reporter gene activity reflects the tissue type and timing of the endogenous gene activity. In a two-component variation, the ammunition vector contains a transcriptional activator, such as yeast GAL4, and a second transgenic construct contains the reporter gene under the control of regulatory sequences that respond to the given transcriptional activator (Brand A H and Perrimon N, Development (1993) 118:401-415). Transposons such as P elements that preferentially insert in the 5′ regions of genes are especially useful for enhancer trapping, since the reporter gene tends to be proximal to the endogenous regulatory elements.

The enhancer trap may additionally include insulator sequences, exemplified by the Gypsy su(Hw) sites in Drosophila, which serve to separate the activity of a gene's enhancer sequences from the corresponding promoter, when inserted between the endogenous gene's enhancer and promoter via a transposon insertion (Roseman R R et al., Genetics (1995) 141:1061-1074).

Alternative preferred sequences encode “gene trap” systems, which are intended to disrupt proper transcription of genes into which they insert. Gene traps are functional when they insert into introns and are usually designed to further provide expression information about the disrupted gene. A variety of gene trap systems have been described (Brennan J and Skarnes W C, Methods Mol Biol (1999) 97:123-138; Zambrowicz B P and Friedrich G A, Int J Dev Biol (1998) 42:1025-1036; Gossler, A and Zachgo J, in Gene Targeting: A Practical Approach (Ed. Joyner A L), Oxford Univ. Press, New York, pp. 181-227). Gene trap vectors typically contain splice acceptor sequence followed by a promoterless reporter gene. Integration of the vector into a transcriptional unit results in a fusion between the endogenous gene and the reporter gene, premature termination of the upstream endogenous transcript, and reporter gene expression in the pattern of the endogenous gene. Gene trap sequences may be especially valuable in transposons such as piggyBac that frequently insert in introns; without the splice acceptor sequences, intronic insertions are frequently spliced out of the final transcript and do not disable the host, even if they are within essential genes.

“Promoter trap” sequences may also be included. Promoter trap vectors consist of a promoterless reporter gene that is activated following insertions in exons of genes, and which directly disrupts the endogenous transcript without a splicing event (Gossler and Zachgo, supra; Zambrowicz, supra).

While the majority of gene trap systems have been designed for mammalian cells, certain P-element vectors have produced aberrant splicing effects by insertion into introns and generation of fusion transcripts that caused premature termination the endogenous transcript (Horowitz H and Berg C A, Genetics (1995) 139:327-335; Goodwin S F et al., Genetics (2000) 154:725-745). A Drosophila gene trap system has been reported (Lukacsovich T et al., Genetics (2001) 157:727-742). Splice trap sequences are further described below.

Construction of the Ammunition Vector

The DNA fragment comprising the ammunition vector is generally inserted into a plasmid vector for replication in bacterial cells and generation of the DNA reagent. All components of the transposable element and the vector backbone are generated and assembled using standard molecular biology methods (Sambrook et al., Molecular Cloning, Cold Spring Harbor, 1989). In addition to the elements that will comprise the ammunition vector, this plasmid must include all the necessary material for replication in bacterial cells. Some appropriate plasmid backbones are PBR322, pUC plasmid derivatives and the Bluescript vector (Stratagene, San Diego, Calif.). In one preferred embodiment, the transposable element also carries sequences that permit replication of the plasmid that contains the ammunition vector, specifically, a bacterial plasmid origin of replication and a drug-resistance marker. These sequences allow plasmid rescue, which is a convenient technique for obtaining a molecular tag to identify the disrupted genes (Hamilton et al., PNAS (1991) 88:2731-2735). Alternatively, the ammunition vector may be inserted into any other appropriate cloning vector, such as bacteriophage lamda derivatives or cosmids.

Optimized Transposable Elements for Generating Lethal Insertions

The invention provides novel ammunition vectors that are optimized for in vivo use and for generating lethal mutations upon insertion.

XP Transposable Elements

In one preferred embodiment, the transposable element is termed an “XP element” and is derived from a P element, specifically an “EP element,” which was engineered for overexpression of Drosophila genes with insertions in their 5′ regions. The EP vector is a modified pCaSpeR4 transformation vector (GI551448), in which Gypsy su(Hw) sites, plasmid rescue sequences, a GAGA-UAS enhancer, and an hsp70 promoter have been inserted in the polylinker, in between the 5′ end of the mini-white gene and the 3′ P end (Rorth P, Proc Natl Acad Sci (1996) 93:12418-12422). In the presence of the GAL4 transcriptional activator, the UAS cassette drives over-expression of flanking genomic sequences.

XP elements have been further optimized in both design and usage to more efficiently mis-express flanking genes, to disrupt transcription of genes with 5′ insertions, and to insert more randomly in the genome of Drosophila hosts. XP elements contain a second “UAS cassette” (i.e., GAGA-UAS-hsp70, as described in Rorth, 1996, supra), oppositely oriented near the 5′ P end, to drive over-expression of sequences on both sides of the inserted element. Subsequent to identification of a lethal gene, overexpression analysis may be use to help characterize the normal function of that gene. The second cassette is flanked by direct repeat FRT recombination sequences that facilitate deletion of the second cassette when the FLP recombinase is expressed in XP hosts (Golic K G, Genetics (1994) 137:551-63). These recombination sequences allow the researcher, by introducing and expressing FLP, to specifically eliminate one UAS cassette and thus determine which direction of misexpression (i.e., on which side of the transposon) is responsible for a phenotype of interest. An exemplary XP sequence is provided in SEQ ID NO:2. While XP elements always retain the w⁺ minigene, the Su(Hw) insulator sequences, and two UAS cassettes, one of which is flanked by FRT repeats, various other modifications are possible. The direct repeat FRT elements may be in either orientation, and they may be either the “long” repeats (GI 172190, nucleotide (nt) 3887-4052), which mediate either inter- or intra-molecular recombination, or they may be “short” repeats (GI 172190, nt 676-723), which only mediate intra-molecular recombination. Additionally, the FRT elements may flank the mini-w⁺ marker gene, as well as a UAS cassette. Plasmid rescue sequences may or may not be included.

We have generated a collection of approximately 9600 unique XP insertion stocks in Drosophila that provides novel data about the usefulness of XP elements. Like EP elements, XP elements contain Gypsy su(Hw) “insulator sequences” that prevent the UAS sequences from acting on the marker gene or on the P element promoter sequence of the P-end (Rorth P, supra; Roseman R R et al., Genetics (1995) 141:1061-1074). Through analysis of the XP collection, we discovered a key benefit of this transposable element, which is that the insulator sequences further enhance the mutagenic potential of su(Hw)-containing elements to generate knock-out alleles, presumably by separating the enhancer and promoter elements of the inserted genes. For generation of the collection, we mobilized XP elements in the female germline. In contrast, P element vectors are traditionally mobilized in the male germline, and EP elements have been mobilized both in the male and female germline. Analysis of the XP collection indicates that this element displays a less-biased insertion profile than the EP element, thus further increasing its utility in the methods of the present invention.

Splice Trap

Splice trap transposons, which disrupt normal splicing and cause premature transcriptional termination when they insert into introns, more efficiently generate gene knockouts than other insertional mutagens. They are specifically useful for intronic insertions, which otherwise are frequently spliced out of the final gene transcript and thus do not disable the host, even if they are within essential genes.

Splice trap sequences minimally comprise a splice acceptor (SA) site, followed by a translation termination codon and a polyadenylation site. These sequences may be encoded by a single exon. As used herein, the “ST 3′ exon” refers to the exon that contains the splice acceptor and translational termination codon, and, if a single exon contains all the splice trapping sequences, the polyadenylation site. As used herein, “splice trapping” and “mis-splicing” refer to the aberrant splicing event whereby an endogenous transcription unit utilizes the transposon-introduced ST 3′ exon preferentially over an endogenous downstream exon. The resulting chimeric mRNA consists of one or more 5′ exons endogenous to the transcription unit fused to the ST 3′ exon. The event causes premature termination of translation and processing at the new polyadenylation site such that the portion of the gene encoded downstream of the transposon is not expressed.

In a preferred embodiment, the splice trapping piggyBac transposon of the present invention utilizes a piggyBac transposable element, which, compared to P elements in Drosophila, is more likely to insert in introns and shows significantly less bias toward particular genomic regions. In another preferred embodiment it contains two ST 3′ exon cassettes, one in each orientation, so that splice trapping will not be dependent on the orientation of the piggyBac insert relative to the mutated gene. As used herein, the orientation of the splice trap element refers to the normal orientation of transcription of the gene from which the ST 3′ exon derives. As used herein, a gene's “terminal exon” refers to the endogenous gene's 3′-most exon. The ST 3′ exon will generally comprise the terminal exon from an endogenous gene of the host system. As further described below, preferred ST 3′ exons usually derive from terminal exons that comprise both a translational termination codon and a polyadenylation signal. However, ST 3′ exons may derive from other exons or may be synthetic.

A key consideration in the construction of the splice trap transposon is the choice of 3′ termination exon (i.e., the “ST 3′ exon”). The following are preferred criteria for the splice trap transposon; these criteria are based on design of a splice trap transposon for Drosophila:
1. The ST 3′ exon, in its normal location should be separated by a large intron (>1 kb). There are many small introns in Drosophila (<70 nt) that have different requirements for splicing than larger introns (Guo M and Mount S M, J Mol Biol (1995) 253:426-437). 3′ exons preceded by such a small intron would not make good a candidate “splice trap”. Data from a piggyBac collection indicates that piggyBac frequently inserts in large (>10 kb) introns. Accordingly, a preferred ST 3′ exon ideally also has a very large intron preceding it.
2. The ST 3′ exon should have good consensus sequences at its endogenous upstream 5′ splice site (5′-ss), its downstream 3′ splice site (3′-ss), its branch consensus, and a pyrimidine rich region at the 3′-ss. It should also have good consensus AAUAAA polyadenylation signal sequence and a 3′ end downstream sequence that has pynmidine stretches (Guo M et al., Mol Cell Biol (1993) 13(2):1104-1118).
3. Amino acid coding capability in all three reading frames is preferred for two reasons. First, this enables the addition of C-terminal epitope tags to each possible reading frame for the identification and characterization of tagged fusion protein expression of the mutated gene. Second, evidence suggests that coding potential of exons is linked to splicing enhancement (Schaal T D and Maniatis T, Mol Cell Biol (1999) 19:261-273). Longer reading frames are further preferred, (see below).
4. A well-characterized 3′ end is preferred. The transcripts of many genes that have been characterized lack molecular characterization of their 3′ ends. Because splice trapping requires a functional polyadenylation site, a 3′ terminal exon that contains a 3′ end that is used in nature—one in which a cDNA containing a poly(A) tail has been identified and characterized and hence has a bonefide 3′ end—is preferred.
5. When the piggyBac transposon is used, a small ST 3′ exon, ideally smaller than 1 kb, is preferred. Since frequency of transposon mobilization generally decreases with increased transposon size, it is preferable to minimize the overall size of the transposable element, and thus the size of the ST 3′ exon, in order to maximize mobilization.
6. The ST 3′ exon should display minimal tissue-specific and temporal regulation, in order to promote mis-splicing in most, and preferably in all tissues throughout development.

For an efficient splice-trapping event, the exogenous ST 3′ exon must effectively compete for slicing with the endogenous downstream splice acceptor of the disrupted transcription unit. Several means can be used to assess the effectiveness of splice trap sequences. In one example, the splice trap transposon is tested in a transient cell or embryo assay. Typically, a plasmid construct is engineered in which the splice trapping transposable element has been inserted within an intron of a test gene. The plasmid is introduced into host embryos or cultured cells derived from the host species. Reverse transcription-polymerase chain reaction (RT-PCR) is used to detect the fusion transcription product between said test gene and the ST 3′ exon and, thus, to determine how efficiently splice-trapping exon promotes mis-splicing.

Alternatively, the splice-trapping transposon can be tested in the host animal by generating multiple independent genomic insertion lines through standard genetics techniques. An exemplary method uses a transposon that has been engineered with recombination substrates, such as FRT sequences, flanking the splice trap sequences. Animals in which the splice trap transposon has inserted in an intron of an essential gene for viability are crossed with animals expressing the appropriate recombinase, such as FLP, which will catalyze removal of the DNA sequence located between the recombination sequences, i.e., the splice-trapping exon. Reversal of the lethal phenotype upon removal of the splice trap sequences provides evidence that splice trapping was responsible for the lethal phenotype. Alternatively, a transposon with a single cassette of splice trap sequences can be used to demonstrate efficient mis-splicing if there are multiple insertions, in both orientations, in the same intron of essential gene. A correlation between the functional orientation (i.e., the splice trap sequences are in the orientations expected to cause mis-splicing) and lethality, provides evidence that mis-splicing occurs and is responsible for lethality. In yet another example, RT-PCR is done from a subset of lines with insertions in introns in order to determine if splicing to the transposon-encoded splice acceptor is occurring.

Modified splice trap transposons provide efficient means to monitor the tissue- and temporal-specific expression of the disrupted gene. In one embodiment, the ST 3′ exon contains an epitope tag (a small stretch of amino acids recognized by commercially available antibodies) in three translational frames preceding the first termination codon and the polyadenylation signal. For instance, a synthetic exon that encodes the myc epitope tag in three reading frames and contains no stop codons has been described (Smith D J, Biotechniques (1997) 23:116-120). The resulting truncated protein will carry an epitope tag fused to its C-terminus, allowing immunohistological visualization of the spatial and temporal expression pattern of the endogenous protein. This splice-trap/tagging transposon is specifically useful for intronic insertions that occur downstream of the translational start codon, and requires that the ST 3′ exon includes some coding sequence into which the epitope tags can be engineered. If an insertion occurs downstream of any protein localization signals, such that the fusion protein contains these signals, the protein's sub-cellular localization may also be detected.

Instead of an epitope tag, the ST 3′ exon may encode a reporter gene, such as GFP, other fluorescent proteins, or beta-galactosidase, which allows direct visualization of the spatial and temporal expression pattern of the endogenous gene product. More preferably, it may encode a transcriptional activator, such as GAL4, which turns on a reporter gene such as GFP in a second transgene. Expression of the reporter gene reflects expression (i.e., transcription) of the endogenous transcription unit and thus provides information about its spatial and temporal expression. This two component system has the advantage of capacity to amplify low or non-detectable levels of gene expression. Said second transgene can be engineered with multiple copies of the responsive elements or promoter sequences, in order to amplify the activity of the transcriptional activator. If the ST 3′ exon encodes a reporter gene or transcriptional activator, reporter gene detection depends on an in-frame fusion to the endogenous gene. Three transposable elements, which encode the reporter or transcriptional activator in three frames, are preferably used to maximize genomic coverage.

In another preferred modification of the splice trap transposon that provides gene expression information, the splice trap sequences are contained in two exons. The first is a modified ST 3′ exon that contains stop codons in three frames but lacks a poly-adenylation signal. The second exon contains the full coding sequence for the reporter or transcriptional activator, as described above, flanked by consensus translational initiation sequences (Kozak) and a 3′ poly-adenylation signal. The two exons are separated by internal ribosome entry site (IRES) sequences, which direct re-initiation of translation of sequences in the second exon. Preferred IRES sequences are subject to minimal tissue- or temporal-specific regulation in the given host. Exemplary IRES sequences derive from the picornavirus virus (Kim D G et al., Mol Cell Biol (1992) 12:3636-3643; Chowdhury K et al., Nucleic Acids Res (1997) 25:1531-1536), human BiP (Yang Q and Sarnow P, Nucleic Acids Res (1997) 25:2800-2807), mouse Gtx (Chappell S A et al., Proc Natl Acad Sci USA (2000) 97:1536-1541) and Drosophila Ubx and Antp (Ye X et al., Mol Cell Biol (1997) 17:1714-1721). This modification obviates the requirements for insertions to occur downstream of the translational start codon as well as the inefficiency inherent in any system that requires a functional fusion between the reporter gene and the endogenous gene.

Introduction of the Transposon into the Host Genome

The ammunition vector may be introduced into the target genome by any expedient methods that permit its integration and stable transmission to progeny animals or cells. “Germline transformation” refers to the stable introduction of an element into the insect host's germ cells; for in vitro uses, “transformation” refers to the stable integration of the element into the genome of a host cultured cell. Typically, if the host organism is an insect, the DNA construct comprising the ammunition vector is introduced via injection. If the host is Drosophila, for instance, a preferred method is injection into pre-blastoderm embryos in close proximity to the developing germ cells (Ashburner, supra, pp 1017-1063). The DNA construct can either be injected with a “helper plasmid,” which transiently supplies the source of transposase but is unable to integrate into the host's genome, or with transposase protein or capped mRNA, or it can be injected into a transgenic host that produces the transposase necessary for the primary transposition. Preferably, if the host produces the transposase source, it is associated with a genetic marker to facilitate its subsequent removal.

Screening Methods

The methods for mobilizing the transposable element, detecting novel insertions, and evaluating whether these insertions are lethal will vary depending on the host organism or cell and the tools available for said host. For any host system, the following criteria should be met: The ammunition vector is efficiently mobilized in the host genome. There are methods for distinguishing the novel insertions from the parental insertions. New insertions are stably maintained in regenerating stocks. There are methods to determine whether each new insertion is lethal to the host cell or organism. The most efficient methods maximize the speed and efficiency of detecting and recovering novel lethal insertions.

In vivo Screens

The screen is most easily performed in a host such as Drosophila, for which efficient genetic tools exist. Key tools are robust marker genes and balancer chromosomes, which minimally contain one or multiple inversions to suppress meiotic recombination between homologues, usually include markers that facilitate crossing schemes by their ease of detection, and usually are themselves homozygous lethal. A “balanced stock” refers to a stock in which mutations of interest are in trans to a balancer chromosome, which suppresses meiotic recombination along the chromosome or chromosomal region containing the mutation. These tools allow the researcher to easily identify animals that harbor novel insertions, to follow the chromosomal locations of these new insertions, to maintain the insertions in stable stocks, and to assess whether individual insertions are homozygous lethal.

The same principles apply to screens in non-model hosts. However, methods for mapping the novel insertions and maintaining the stocks generally rely primarily upon introduced, as opposed to endogenous markers, or use molecular methods, such as PCR, to detect insertion-based polymorphisms.

Experimental methods for mobilization of the ammunition vector in an insect typically involve an initial germline transformation into a limited number of recipient organisms and subsequent germ-line mobilizations, which generate large numbers of progeny bearing individual insertions in different chromosomal loci. To disperse the element to new chromosomal loci, a genetic cross typically brings together the ammunition vector with a source of transposase, causing mobilization of the element in the germline of animals that harbor both elements. For use in Drosophila, for instance, well characterized strains exist that contain the P element or piggyBac transposase. The “delta 2-3” transgene provides an integrated, stable source of P-element transposase (Robertson H M et al., Genetics (1988) 118:461-70). We have generated an integrated constitutive source of piggyBac transposase (contained in nt 334-2451 of SEQ ID NO:1; open reading frame corresponds to nt 334-2122 of SEQ ID NO:1) under the control of the Drosophila alpha1-tubulin 5′ UTR (nucleotides 45898-46694 of P1 DS00464, gi3293209; α1-tubulin gene described in gi158730) and K10 3′ UTR (nucleotides 21925-23645 of cosmid 30B8, gi3928153; K10 gene described in gi8148), referred to as “P[a-tub:pBac]”. Alternatively, the animals that carry the ammunition vector may contain a regulated form of the transposase, for instance under control of a heat shock promoter, to mobilize the ammunition vector in the parental host but not in their progeny bearing new insertions.

Following germline transformation of host animals, one or a few insect lines that contain the ammunition vector in genetically characterized chromosomal locations are selected as parental hosts; the ammunition vector will be further mobilized in their germline cells. The choice of parental host is a key determinant of how progeny bearing novel insertions are selected. In a preferred application in Drosophila, the ammunition vector is initially transferred to a dominantly marked balancer chromosome. Segregation of the primary marker from that associated with the balancer chromosome will identify progeny with novel insertions. Alternatively, in a host such as Manduca sexta, for which balancer chromosomes do not exist, the ammunition vector may be sex-linked. For instance, if an X-chromosome element is mobilized in males, all male progeny that contain the ammunition vector contain autosomal and thus new insertions, since their X-chromosomes will have derived from non-host mothers (except in rare instances of Y-chromosome insertions). In yet another application, the ammunition vector contains enhancer trap sequences, and the screen for new insertions monitors expression of the reporter gene. In this case, expression pattern of the parental host is characterized prior to mobilization. The novel insertions are identified in progeny that express the marker gene in a different pattern. An exemplary screening strategy using these methods is provided in the examples. Finally, when genetic methods for distinguishing the novel insertions are unavailable, molecular methods may be used. For example, inverse PCR can be used to isolate and analyze genomic DNA flanking the transposon (Dalby B, Genetics (1995) 139:757-766). The DNA fragments are then sequenced by standard means to generate “sequence tags,” which are typically 50-300 base pair (bp). The sequence tag defines the insertion site and distinguishes the novel insertions from parental insertions. For organisms with sequenced genomes, comparison of the flanking sequence tag with genornic sequence identifies the gene most likely affected by insertion.

A series of crosses effects the generation and selection of progeny with novel insertions. Typically, a first cross is used to introduce a transposase source and mobilize the ammunition vector in progeny of the parental host. Dysgenic progeny animals (ie., animals in which transposable elements are actively transposing) are selected; they are generally identified as those that display marker genes associated with both the ammunition vector and the transposase. Dysgenic animals are out-crossed in order to segregate the source of transposase from the ammunition vector, which allows for the recovery of stable novel insertions.

The subsequent crosses are designed to evaluate whether the novel insertions are lethal, and these crosses may serve additional functions. In a preferred screen in Drosophila, the identification of lethal insertions coincides with the mapping and balancing of the novel insertions, which requires one or a few genetic crosses. Strategies for mapping and stocking dominant mutations and genetically equivalent marked transposon insertions are based upon principles of Mendelian segregation and are well known to those skilled in the art (e.g., Fly pushing: The Theory and Practice of Drosophila melanogaster Genetics (1997) Cold Spring Harbor Press, Plainview, N.Y., pp. 49, 63). If the resulting balanced stock never produces adults homozygous for the insertion and therefore lacking the dominantly marked balancer chromosome, the novel insertion is lethal.

In an exemplary screen in a non-model host, individual novel progeny are isolated and outcrossed to wild-type or other non-host animals in order to found a stock, which is maintained by the continual selection for animals that display the primary marker. Some of the progeny from each outcross, which are heterozygous for the insertion, are crossed to their siblings to identify the lethal insertions. Lethality is assessed according to principles of Mendelian segregation. If the insertion is homozygous viable, approximately three-quarters of progeny should display the primary marker. Two quarters will be heterozygous for the ammunition vector—half of these having received the vector from their mother and half from their father—one quarter will be homozygous for the vector, and one quarter will be homozygous for the non-inserted chromosome. If the insertion is homozygous lethal, only two-thirds should display the primary marker, reflecting the absence of the class homozygous for the ammunition vector. In an alternative or supplemental method, the researcher observes progeny of the crosses between heterozygous siblings for evidence of dying embryos, larvae, or pupae that display the primary marker. For this method to give reliable indication of lethality, the researcher should be able to discern significant numbers of dying and dead progeny, all of which must display the primary marker. The researcher can instead use molecular methods to determine if homozygous animals are ever produced in the stock. Methods such as inverse PCR or plasmid rescue are used to generate a sequence tag. Multiple PCR-based methods are available and well known in the art. For instance, the flanking sequence tag can be used to design and generate PCR primers on both sides of the insertion, which can be used to amplify a wild-type gene fragment. Amplification from the transposon-containing chromosome will either produce a larger fragment, or, if the transposon is large and the PCR extension time short, will not amplify a detectable band. If amplification reactions from a large number of individual animals in the stock always produce the wild-type band, it can be concluded that the stock contains no homozygous animals.

Even if some homozygous adults are detected, the insertion may still be significantly deleterious as to indicate an appropriate target. Additional experiments may test homozygous animals for fecundity, locomotion, eating habits, or other essential functions; stocks in which the insertion disrupts any of these faculties may contain insertions in candidate pesticide targets. If the screen uses Drosophila, animals that lack the balancer chromosome are analyzed for such defects. If the screen uses a non-model host, all progeny from an inter se cross (i.e., a cross between genetically equivalent individuals) that display the primary marker are analyzed for such defects. If individuals from a stock consistently display a relevant phenotype, they are tested by the aforementioned genetic or molecular means to determine whether they segregate with homozygous insertion animals.

In vitro Screens

A screen performed in the animal host depends upon methods for analyzing the effect of the inserted chromosome when homozygous. If the screen is performed in cultured cells (“in vitro”), there may be no straightforward means to generate cells homozygous for the transposon insertion. In this case, induction of ectopic transcription of genomic sequences is used to dominantly disrupt function of genes with transposon insertions.

In this aspect of the invention, the ammunition vector is engineered with heterologous gene regulatory elements, specifically promoters and/or enhancers, designed to direct ectopic transcription of genomic DNA flanking the ammunition vector. Exemplary regulatory sequences include the metallothionein promoter, which responds to the addition of copper to the growth media, (Bunch T A et al., Nucleic Acids Res (1988) 16:1043-1061), the ecdysone responsive promoter, which responds to addition of hormone (No D et al., Proc Natl Acad Sci USA (1996): 93: 3346-3351), and a mutated hormone binding domain from mouse estrogen receptor, which responds to the addition of 4-hydroxytamoxifen (Littlewood, T D et al., Nucleic Acids Res (1995) 23:1686-1690).

In a preferred embodiment, a panel of cells lines containing different, random transposon insertions is generated. The panel may be generated by transforming a parental cell line with the ammunition vector, and inducing expression of a transposase in the parental cell line (e.g., by transiently transfecting a transposase or by activating an inducible transgene) to randomly mobilize the transposon in progeny cells. Alternatively, the ammunition vector is introduced to cultured insect cells by transfection and is accompanied by a construct containing the transposase, which is required for efficient integration into the cellular genome. Cells from the Drosophila embryonic cell line Kc167 have been stably and efficiently transformed with P elements by transfection with plasmids containing the transposon and the transposase (Segal D et al., Somat Cell Mol Genet (1996) 22:159-165). P elements inserted in single copies per locus, at approximately 1-50 copies per cellular genome. Using similar methods, we have mobilized piggyBac elements in cultured Drosophila Schneider 2 (S2) cells. We have shown that when a transposon-containing plasmid is transfected without accompanying transposase, gene sequences contained by the transposon are transiently expressed in over half of the cells, but the number of expressing cells drops to less than one percent after several weeks. In contrast, in the presence of transposase, expression levels remain significantly higher over that period, due to insertion of the transposon into the cells' genomes.

Clonal cell lines are derived from individual progeny with novel transposon insertions. They may be recovered by plating progeny cells at clonal concentrations. Alternatively, individual progeny cells may be recovered by cell sorting methods (e.g., Tanke H J and van der Keur M, Trends Biotechnol (1993) 11:55-62; Bryant Z et al., Proc Natl Acad Sci (1999) 96:5559-5564). Cell lines are typically kept in multi-well plates, or any other system that facilitates handling of hundreds or thousands of lines. Cell lines are expanded, and the panel is replicated. One replica is preserved for recovery of the interesting cell lines, while the other (“test panel”) is assayed for insertions in essential genes. Using the test panel, ectopic transcription is induced from the gene regulatory elements in the ammunition vector, for instance, by adding metal or hormone to the media to induce transcription from, respectively, the metallothionein or ecdysone responsive promoter. After a few days the cell lines in the test panel are observed for defective growth and/or reduced viability. A preponderance of dead cells, or a significantly reduced number of cells compared to the corresponding line in the reference panel indicates that ectopic transcription from the inserted transposon is deleterious to the cell, and identifies a candidate lethal gene. Cell lethality can be assayed by any convenient assay, such as trypan blue staining.

Dominant loss-of-function is expected from both protein-based and RNA-based effects. The protein-based effect will occur when an insertion into the middle of a gene produces a truncated protein that interferes with the normal function of a wild-type copy.

It is furthermore expected that a proportion of insertions will not produce ectopic proteins but will generate transcripts that act dominantly at the mRNA level to inhibit functional expression of the wild type copy of the gene. Ammunition vectors that insert downstream or in the middle of coding sequences of genes can produce, respectively, full-length or partial antisense transcripts. Experimental evidence has shown that ectopic transcripts can specifically inhibit targeted genes. The introduction of single stranded antisense RNA molecules, either by direct injection or by a transgenic element, has produced reduction of function phenotypes for several Drosophila genes (e.g., Schuh and Jackle, Genome (1989) 31:422-425; Patel R and Jacobs-Lorena M, Developmental Genetics (1992) 13:256-263). More recently, the introduction of double stranded RNA (dsRNA) has been used to disrupt gene function by degradation of cognate mRNAs (Kennerdell J R and Carthew R W, Cell (1998) 95:1017-1026; Zamore P D et al., Cell (2000) 101:25-33). When dsRNA is introduced, the entire transcript need not be represented; fragments representing a minority portion of the endogenous gene's coding sequence can produce loss-of-function phenotypes that are indistinguishable from the phenotypes produced from the full-length antisense transcripts (see Misquitta and Paterson, Yang et al). Thus, transposable elements that insert in the middle of genes and overexpress only a portion of the antisense coding sequences should still inhibit gene function. Although there have been published reports claiming that single-stranded antisense RNA does not efficiently produce an RNA interference (RNAi) effect, we have shown that, in vivo, EP and XP insertions in coding sequences can dominantly disrupt gene function.

Insertions that act at the protein level can be distinguished from those that act at the RNA level by addition to the media of an agent, such as cycloheximide, which inhibits protein synthesis. Inhibiting protein synthesis should eliminate lethal phenotypes caused by protein overproduction.

Moreover, inducing transcription from some insertions will yield dominant positive effects, which occur when ectopic transcription leads to production of a functional protein. Lethality associated with dominant positive effects may indicate that the overexpressed gene is an appropriate target for agonist pesticides. Insertions that cause loss of function of the endogenous gene can be confirmed by RNAi validation in wild type cells, as described below.

Identification of the Disrupted Gene

Various molecular biology techniques can be used to characterize the genomic sequence flanking the ammunition vector and identify the disrupted gene. In a preferred embodiment, inverse PCR is used to amplify a short molecular tag, which then serves as the template for sequencing the fragment. Alternatively, if the ammunition vector contains the requisite components for bacterial plasmid replication, plasmid rescue techniques may be used clone the abutting DNA fragments (Hamilton et al., supra).

Biological Array

In one preferred embodiment, screening methods are used to generate a “biological array,” used herein to refer an indexed collection of transgenic insect lines or insect cell lines, the genome of each containing at least one (most preferably only one) introduced transposable element that inserts (mutates) one of the insect's genes, such that the complete collection contains a mutation in essentially every gene in the host's genome. In a further preferred embodiment, the array comprises piggyBac insertions. In a preferred application in Drosophila, the array further comprises XP insertions. Each member of the array is a stable, regenerable stock consisting of animals or cell lines that contain an insertion of a transposable element. Each member of the array is characterized with respect to the genomic DNA flanking the insertion and indexed according to flanking sequence, mutated gene, and/or chromosomal position.

As used herein, the term “contains a mutation in essentially every gene” refers to the statistical situation where there is generally at least about a 70% probability that the genomes of insect or cell lines of the array collectively contain at least one transposon insertion in any gene of the genome, more preferably at least an 85% or 95% probability, as determined by standard statistical methods. For the purposes of determining whether genes comprise transposon insertions, the term gene refers to the genomic sequence between start and stop codons, and may include upstream and downstream regulatory sequences, such as promoters, enhancers, 5′ and 3′ untranslated regions, polyadenylation signals, etc. For instance, for the purpose of determining genome saturation of a piggyBac collection, we defined a gene as tagged (i.e., containing a transposon mutation) if a transposon inserted between the start and stop codons or was within 1,000 bp of the start codon

The most efficient arrays are generated in hosts for which genomic sequence exists. Ordered genomic sequence allows the researcher to quickly map the novel insertions, to determine the location of an insertion within a gene or predicted gene, and to eliminate redundant insertions when multiple insertions disrupt the same gene. Genomic coverage preferably exists at coverage of at least 0.5×, more preferably at least 1×, 2×, or 5×. Genomic sequence may be obtained through any available methods, such as a clone-based physical map approach or the whole-genome shotgun approach; these were used for sequencing, respectively, C. elegans and D. melanogaster genomes (The C. elegans Sequencing Consortium, Science (1998) 282:2012-2018; Myers E W et al., Science (2000) 287:2196-2204).

If whole genome sequence is not available, relevant genomic sequence data may be generated as insertions are characterized. For instance, generating whole genome sequence may not be biologically feasible for host organisms that have large genome sizes due to a preponderance of repetitive DNA sequences.

Alternatively still, a collection of full or partial cDNA sequences can form the basis for ordering transposon insertions. Methods for generating cDNA sequences and expressed sequence tags (ESTs) are well known in the art. Exemplary methods for using ESTs to generate a collection of Drosophila cDNA sequences are provided by Rubin et al. (Science (2000) 287:2222-4). The cDNA sequence is preferably of sufficient coverage and has sufficient annotation to represent mostly whole genes. While transposons such as piggyBac frequently insert in introns, which are unrepresented in cDNA, sequence tags from flanking genomic DNA may still include cDNA sequences, depending on where in a given intron the transposon inserts, how long the sequence tag is, and how large the intron is. While cDNA sequence does not allow an ordered array of transposon insertions, it can provide sufficient information to generate a generally non-redundant array that mutates essentially all genes in the host genome.

Characterization of Lethal Genes

Preferred methods for the generation of transposon insertions usually produce single insertions in each host cell or animal. However, some individuals may harbor multiple ammunition vectors, complicating analysis. If the host is an insect, standard genetic crosses are used to segregate the various elements and the “sub-progeny” are retested to determine which insertion is recessive lethal. Since genetic crosses are not available to cultured cells, the most efficient method to identify which one of multiple insertions in a cell line induces lethality is to use RNA interference (RNAi) techniques. Double stranded (ds)RNA corresponding to candidate sequences are introduced to wild-type cells to induce specific loss-of-function phenotypes (Hammond S M et al., Nature (2000) 404:293-296).

Further computational, molecular biology and genetic assays as are known in the art can confirm that the candidate sequences correspond to lethal genes. A first step is characterizing the putative disrupted gene from flanking sequence. Typically, the tagged sequence is subjected to BLAST analysis to determine whether the flanking sequence corresponds to or contains substantial homology to known genes (Altschul et al., J. Mol. Biol. (1997) 215:403-410; http://blast.wustl.edu/blast/README.html). When it does not, computer programs such as GENSCAN are used to predict which flanking sequences encode proteins (Burge C and Karlin S, J. Mol. Biol. (1997) 268:78-94).

Sometimes, other non-insertional mutations occur during the transposable element mobilization and these can confuse matters. Certain transposons, such as the P element, may imprecisely excise and cause chromosomal aberrations distinct from the insertion site (termed “second site lethals”). So-called “hit and run” events occur when remobilization follows an initial mutagenic event. Various approaches can corroborate the relationship between the insertion and the lethal mutation. If the insertion is responsible for lethality, flies heterozygous for the inserted chromosome and a homologous chromosome deficient for a genomic region encompassing the insertion site should also exhibit the recessive lethal phenotype. If animals carrying this chromosomal combination die, this at least localizes a lethal mutation to this region. If they survive, the lethal mutation lies elsewhere in the genome. Alternatively, exposure to the appropriate transposase and subsequent excision of the inserted element (as determined by genetic or molecular means) will allow the experimenter to validate that the insertion was the true source of lethality. If excision does not revert the phenotype, it remains a possibility that the excision itself produced a molecular lesion, in which case it will be useful to molecularly characterize the region of the insertion, such as by PCR or sequencing methods, according to any available method. Finally, a wild type version of the putative lethal gene, termed a rescue construct, may be cloned and re-introduced into the host. This can be derived from a genomic clone carrying regulatory and coding sequences of the gene, from a fusion of the genomic regulatory sequences of the gene and cDNA of the coding region, or from a fusion of heterologous regulatory sequences and cDNA of the coding region. If the insertion is lethal, this transgene should rescue or significantly improve the phenotype of the test animals, which are homozygous for the insertion and carry the wild-type transgene.

It will often be desirable to confirm that the insertion reduces or eliminates transcription of the normal gene product. This analysis may be dispensable if the genetic and computational analysis have confirmed that the lethality maps to the insertion site, that the insertion disrupts confirmed coding sequence and would obviously result in a truncated protein. The analysis will be especially relevant if the ammunition vector appears to be in an intron or other non-coding sequence, and it is unclear how the insertion would affect the normal transcription and splicing. Several techniques for analysis of mRNA, such as Northern blotting, slot blotting, ribonuclease protection, quantitative RT-PCR, and microarray analysis are available and well known to skilled practitioners (e.g., Current Protocols in Molecular Biology (1994) Ausubel F M et al., eds., John Wiley & Sons, Inc., chapter 4; White K P et al., Science (1999) 286:2179-2184; Freeman W M et al., Biotechniques (1999) 26:112-125; Bryant Z et al., Proc Natl Acad Sci (1999) 96:5559-5564). Northern blot analysis comprises preferred methods. For in vivo applications, northern analysis, using mRNA from transgenic host and wild type or other non-host animals and probes made from the candidate sequences, can determine whether flanking sequences correspond to disrupted genes. Depending on the position of the insertion, the disrupted gene is expected to produce a significantly reduced or eliminated transcript compared to non-host, or a differently sized transcript, representing a chimeric fusion with transposon sequences. If the insertion disables but does not kill the host, homozygous insertion animals can supply mRNA for the blot. If the gene is recessive lethal, it still may be possible to collect homozygous embryos or larvae that supply the mutant transcript. For instance, some balancer chromosomes contain markers that allow detection early in development. In Drosophila, for instance, such balancers include the “TM6” third chromosome balancer chromosomes, marked with the Tubby endogenous dominant larval/pupal marker (Lindsley D L and Zimm G S (eds.) The Genome of Drosophila Melanogaster, 1992, Academic Press, New York), or balancers that are marked with lacZ or GFP (Casso D et al., Mech Dev (1999) 88:229-32). Animals in which the insertion is in trans to such balancers are obtained by standard methods and crossed to each other. The homozygous progeny that do not display the balancer marker can be collected. It is further possible to compare mRNA from heterozygous insertion animals to mRNA from wild-type animals; quantification of the signal should reveal a reduction in one copy of the gene. An alternative preferred method is to perform quantitative RT-PCR, for instance, using the Taqman® system (Applied Biosystems, Foster City, Calif.; Gelmini S et al., Clinical Chemistry (1997) 43:752-758). In situ hybridization techniques, performed on fixed tissue sections or whole mount tissues, embryos or animals, offer alternative means for assessing expression levels (Tautz D and Pfeifle C, Chromosoma (1989) 98:81-85).

Similar kinds of analysis may be performed for in vitro applications, when it is expected that RNA-mediated effects cause a loss-of-function phenotype. It has been shown that RNAi functions by degradation of the cognate cellular mRNA (Zamore et al., supra; Hammond S M et al., Nature (2000) 404:293-296). Accordingly, quantitative RT-PCR analysis, such as using the Taqman® system, is a preferred method determining whether an insertion reduces or eliminates transcription of the normal gene product.

A third step is independent confirmation that inhibition of gene activity will be lethal to the animal. RNAi techniques are preferred methods. Wild-type animals are analyzed with double dsRNA directed towards the candidate sequences in order to specifically disrupt the endogenous gene (Kennerdell J R and Carthew R W Cell (1998) 95:1017-1026; Clemens J C et al., Proc Natl Acad Sci (2000) 97:6499-503). Alternatively, homologous recombination can be used to create a de novo insertion in the candidate gene of wild-type animals (Rong Y S and Golic K G, Science (2000) 288:2013-2018). For potential targets that were identified in vitro, it is important to confirm that loss of function is lethal to the intact organism as well.

Identification of Orthologous Proteins

When a novel lethal gene is isolated from a screen using a model organism, such as Drosophila, it will often be useful to develop biochemical assays using the orthologous protein from an actual pest species. Such assays are used to find molecules that can inhibit the protein target. While the orthologs of the model and pest insects may have essentially the same function, differences in their protein structure will affect properties such as interactions with other proteins, compound binding and stability. Thus, results of the biochemical assays are typically meaningful only for a specific protein to be targeted. In general, orthologs in different species retain the same function due to presence of one or more protein motifs and/or 3-dimensional structures. Methods of identifying the orthologs are known in the art. Orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10:1204-1210). Programs for multiple sequence alignment, such as CLUSTAL (Ibompson J D et al, 1994, Nucleic Acids Res 22:4673-4680) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs.

When sequence databases are not available, nucleic acid hybridization methods substitute for computational analysis to find orthologous genes. If a screen utilizes a model host, instead of the actual pest, sequence information for this host will almost always be available, whereas sequence information for the actual pest might not be available. Degenerate PCR and library screening are common methods for finding related gene sequences and are well known to those skilled in the art (e.g., Current Protocol in Molecular Biology, Vol. 1, Chap. 2.10, John Wiley & Sons, Publishers (1994); Sambrook et al., (Molecular Cloning (1989), Cold Spring Harbor, Chapter 8). An exemplary method involves generating a cDNA library from the organism of interest and probing the library with partially homologous gene probes. After successful amplification or isolation of a segment of a putative ortholog, that segment may be cloned and sequenced by standard techniques and utilized as a probe to isolate a complete cDNA or genomic clone. Alternatively, it is possible to initiate an EST project to generate a database of sequence information for the host. Once the candidate ortholog(s) are identified by any of these means, candidate orthologous sequence are used as bait (the “query”) for the reverse BLAST against sequences from the screened host.

If assays are developed for the orthologous pest target, the researcher should also confirm that disruption of this gene kills or disables the intended pest using above-described methods.

Characterization of Pesticide Targets

Many criteria help determine whether a novel lethal gene is a suitable pesticide target. The following characteristics are preferred criteria for pesticide targets. An ideal target is broadly required throughout development so that inhibition at any stage of the life cycle will kill the pest; the “lethal window” refers to the time period when inhibiting a particular protein target will kill the pest. The target should be dosage sensitive, so that even a partial inhibition of function will kill the pest. For field or greenhouse crops, target inhibition should quickly kill the pest (“rapid knockdown”) to minimize damage. The target should be as divergent as possible from orthologous genes in species intended not to be harmed by the pesticide, such as vertebrates, beneficial invertebrates, or plants. The target should encode a protein with a known function for which a robust assay can be readily designed. For any specific application, these requirements will vary. For example, control of disease vectors or urban pests such as cockroaches or termites often emphasizes safety over speed. In this case rapid knockdown or broad expression are not as important in target selection. In order to identify and validate genes as pesticide targets a several step process demonstrates that a potential target has the desired characteristics before embarking on the time- and money-intensive steps of creating an assay and screening for chemical antagonists of target function.

Since the most preferred targets do not have very closely related orthologs in non-targeted species, a search for orthologs using available sequence databases as described above, is generally performed. If a putative target has closely related orthologs in other species, this does not disqualify it. Small differences in protein structure can affect the ability of small molecules to modulate the protein, and different organisms' physiology can affect their response to a compound. The discovery of closely related orthologs may, however, affect the prioritization of a potential target, and may guide future assay development and analysis.

The first and easiest step to defining the lethal window is determining the expression pattern of a candidate target gene is a first step toward its requirement, and expression information is often among the most easily obtained data. If the target is not expressed it cannot be interfered with by the pesticide. Generally a target should be expressed as broadly as possible to maximize the opportunities to kill or disable the pest. However, narrower expression windows are also feasible. Suitable targets may be expressed when the pest is directly causing economic damage, such as a larval feeding stage for most crop pests, or at stages when the pest is accessible to pesticide treatment, such as migratory stages or stages when the insect is not concealed within the plant. Gene expression can be monitored at either the mRNA or protein level.

Taqman® analysis, as described above, is a preferred means to detect mRNA expression and allows analysis from individual tissues. Northern blotting and in situ hybridization are alternative means. Northern blotting is useful for obtaining a temporal expression profile from a variety of developmental stages. In situ hybridization has the advantage of showing the spatial expression pattern but may be more labor intensive.

Similarly protein expression can be monitored by a variety of means including transfer of extracted protein to an immobilizing matrix (Western blotting) or by in situ detection in whole mount preparations or sections (Harlow E and Lane D (eds.) Using Antibodies: A Laboratory Manual, 1999, Cold Spring Harbor Laboratory Press, New York). Proteins are most commonly detected with specific antibodies or antisera directed against either the protein or specific peptides. Western blotting is extremely sensitive and provides a quantitative measure of protein levels. However, it detects protein at a tissue or organismal level and provides no information on distribution of the protein within a tissue or cell. In contrast, in situ detection of protein can provide this information, revealing the cellular or subcellular distribution of a potential pesticide target. Protein expression can be directly visualized in the living animal by creating a transgenic animal expressing a fusion gene comprised of the coding sequences of a reporter such as GFP under control of the endogenous gene's regulatory sequences; the reporter may additionally be fused to the coding sequence of the endogenous gene. A transgenic reporter construct can also comprise an endogenous gene's cDNA fused to sequences encoding an epitope tag, a small stretch of amino acids recognized by an existing antibody or anti-sera, often available through commercial sources, which allows visualization by in situ antibody detection methods.

Ideally a pesticide target should be essential for organismal viability at all stages of the lifecycle. While a broad expression profile suggests a broad temporal requirement for the gene, this necessity may also be tested directly prior to initiating assay development. Since animals with mutations in essential genes die or arrest at the first point in their development at which zygotic activity of the gene is required, determining requirements for the gene later in development may be complicated.

RNAi techniques, which can be used to silence specific genes by introduction of dsRNA corresponding to sequences of the targeted gene, offer means for testing the developmental requirements for a gene (Kennerdell and Carthew, 1998, supra). A system for producing heritable, inducible RNAi phenotypes in Drosophila has been described (Kennerdell J R and Carthew R W, Nature Biotechnology (2000) 17: 896-898). Briefly, a transgenic construct is used to generate a hairpin dsRNA under transcriptional control of yeast UAS regulatory sequences. The GAL4 transcriptional activator can be expressed under a heat-shock promoter, to allow expression at any developmental time, or under a temporal-specific promoter element. Inactivation of gene function should closely follow activation of the dsRNA hairpin. There have also been unpublished reports suggesting that feeding dsRNA to Drosophila can produce loss-of-function phenotypes, similar to RNAi feeding methods available for C. elegans (Timmons L and Fire A, Nature (1998) 395:854). As such methods become available for insects, they may represent the most efficient method of testing the temporal requirements for putative target genes.

An alternative solution to this problem is to use transgenic conditional or regulatable genes to control expression to simulate disruption a putative target gene at various times in development. There are a variety of tools for this purpose available in Drosophila, and many should be functional in other insect hosts. These methods involve introducing a transgenic rescue construct into a homozygous mutant background, using standard transformation techniques and genetic crosses, and turning off the rescuing gene at various points in development. In each of the cases described below, the coding region of the gene of interest is cloned into the appropriate transformation vector and the resulting construct is introduced into transgenic animals.

Preferred systems are two component, modular control systems that rely on gene regulatory elements adapted from fungal or bacterial species.

The tetracycline- (Tc-) mediated system is an exemplary two-component system. The “tet transactivator (tTA)” and “tet operator (tetO)” comprise, respectively, the transcriptional activator and the responsive promoter sequences The tTA protein binds to and activates transcription from the tetO sequences only in the absence of tetracycline, requiring the systemic administration and subsequent removal of tetracycline in order to turn on gene expression (Gossen M and Bujard H, Proc Natl Acad Sci (1992) 89:5547-5551). The system, which has been adapted for Drosophila applications (Bello B et al., Development (1998) 125:2193-2202), can allow regulated rescue of the lethal gene. For this use, the candidate gene is under the control of the tetO sequences and the two components are introduced into a genetic background homozygous for the lethal insertion. The tTA protein is driven by a strong and relatively ubiquitous promoter, such as the armadillo promoter, which drives ubiquitous expression in the embryo and in larval discs (Sanson B et al., Nature (1996) 383:627-630), or by the regulatory sequences of the lethal gene itself. The addition of tetracycline to the food at various developmental time points should extinguish expression of the rescuing sequence. If the addition of drug kills the animal, the result demonstrates a requirement for the gene at the particular stage.

Alternatively, the Gal4/UAS system, derived from yeast, utilizes the GAL4 transcriptional activator to bind to UAS enhancer sequences and initiate transcription of nearby coding sequences (Brand A H and Perrimon N, Development (1993) 118:401-415). When used to misexpress a particular gene in vivo, transgenic insect lines are generated that contain the gene of interest operably fused to a UAS promoter and a heterologous minimal promoter, such as that from the Drosophila hsp70 gene (Huynh C Q and Zieler H, Journal of Molecular Biology (1999) 288:13-20). When these UAS “target” lines are crossed with GAL4 “driver” lines that contain a tissue-specific source of GAL4, resulting progeny mis-express the gene of interest in the specific pattern of the GAL4 transgene. The system may be used in to test the temporal requirements of a recessive lethal gene. Rearing the relevant stocks at 29° C. promotes mid levels of expression, which may permit rescue of the lethal phenotype. If it does, the animals may be removed to the restrictive temperature (18° C.) at various developmental times to test the requirements for the putative target at these times.

The yeast FLP/FRT system relies on a site-specific DNA recombinase to exert its action (Golic K G and Lindquist S, Cell (1989) 59:499-509). By engineering the target sites (FRTs) for the recombinase into a gene, it can be either turned on or off. The expression of the FLP enzyme therefore serves as a switch to control expression of the target gene, which is under the control of either its endogenous promoter or a heterologous promoter. For example, two FRTs flanking the coding region of the gene will inactivate the gene when the recombinase is expressed. An inducible promoter, such as the hsp70 promoter, or tissue or temporally specific promoter or regulatory element can control FLP expression. Inactivation of the target gene will follow activation of the FLP recombinase.

Expression of the rescuing transgene can also be controlled via single-component systems that rely on the host's endogenous gene regulatory sequences. In Drosophila, for instance, transcription can be controlled by placing the candidate gene under the control of an inducible promoter such as the Hsp70 promoter or the heavy metal inducible metallothionein (mt) promoter. Expression of the target gene is then directed by the appropriate stimulus (heat shock or heavy metal treatment) and the gene can be turned on or off at will. These systems are convenient but suffer from several problems: they can not be restricted in expression to specific tissues and they are expressed at low levels even without induction.

Since these systems for conditional or regulatable gene expression function at the level of transcription, they depend on protein turnover to reduce target gene function. There are alternative methods to disrupt gene function at the protein level. Existing dominant negative alleles of the endogenous gene may be expressed in a conditional or regulated manner to antagonize endogenous gene function. Studies of homologous proteins may indicate means to engineer temperature sensitive mutations, which may be incorporated into a rescue construct. Transgenic animals that express such a construct and are mutant for the gene of interest may be shifted to the restrictive temperature, at various stages, in order to define the lethal window.

Finally, it remains an option to bypass the genetic experiments that test the candidate gene's requirement, and to test this directly by proceeding directly to screening, generally high throughput screening methods, as described below, to identify lead antagonist compounds, and to use these to inhibit gene function at various developmental stages.

The same methods may be used to test a target's potential for rapid knockdown and for partial inhibition to kill or disable the insect. Inducible gene repression systems, either inducible RNAi or inducible transgenic systems, offer the best means to test rapid knockdown. Preferably, introduction of the inactivating condition, such as feeding or inducing inhibitory RNA, or removal of a condition that allows expression of a rescue construct in the mutant background, induces lethality within 24 hours, more preferably within ten hours, and most preferably within three hours.

In practical terms it is impossible to deliver enough pesticide to an organism to totally inhibit the function of the target. Thus pesticide targets must not only be essential for viability, but a substantial fraction of the normal activity level should be essential for viability of the organism. In general, good pesticide targets are required at levels at least several percent of normal; if the insect survives when, for instance, greater than 95% of target gene function is inhibited, it will be difficult or impossible find efficient pesticides that antagonize the given target. However, for most genes, inhibition of greater than 50% is required to kill or disable the host. Thus it is expected that for most preferred targets, there is a range of approximately 70% to 95% inhibition (i.e., approximately 5% to 30% of gene function persists) that is required to kill the host. A preferred way to test the dosage sensitivity of a target is by the Tet system described above. When these constructs are used to provide target expression in animals otherwise lacking the target gene, the minimum level necessary for survival can be determined by varying the concentration of tetracycline in the diet and then measuring the corresponding levels of target protein by the methods described above.

While the above set of criteria pertains to the function of the potential target within the insect, a second set pertains to the biochemical and pharmacological characteristics of the putative target protein. Preferred targets for assay development are those for which a function is known or may be assumed based upon homology to known genes or proteins. Most preferred pesticide targets encode proteins with small, well-characterized active sites, and belong to families for which there is precedence for chemical or biological modulation. Pesticides typically disrupt an active site, pore, or allosteric or regulatory site. While less common, pesticides may also inhibit protein-protein interactions. Exemplary targets are enzymes or soluble proteins with ligand binding sites, including protein kinases, protein phosphatases, proteases, protease inhibitors, topoisomerases, helicases, polymerases, phosphodiesterases, phospholipases, prolylisomerases, nuclear hormone receptors, GTPase activating proteins (GAPs), and guanine nucleotide exchange factors (GEFs), and a range of metabolic enzymes. Alternative exemplary targets are membrane proteins, such as G protein coupled receptors (GPCRs), protein kinase receptors, ligand-gated ion channels, voltage dependent ion channels, transporters, and so forth.

Identification of Pesticidal Agents

Assay Development

Described herein are methods (screening assays) to identify agents that modulate the function of pesticide targets. In a preferred embodiment, pesticidal agents inhibit the activity (including transcription, protein expression, protein localization, and cellular or extra-cellular activity) of identified targets. Agonist agents may also be identified. Pesticidal agents generally act directly on the protein products of target genes. However, they may also modulate protein function indirectly by acting at the nucleic acid (typically mRNA) level.

Screening assays for pesticidal agents may be cell-based (using live cells, dead cells, or a particular cellular fraction) or may use a cell-free system (using substantially or partially purified protein, or crude cellular extracts) that recreates or retains the relevant biochemical reaction of the target protein (reviewed in Sittampalam G S et al., Curr Opin Chem Biol (1997) 1:384-91 and accompanying references). Screening assays may detect a variety of molecular events, including protein-DNA interactions, protein-protein interactions (e.g., receptor-ligand binding), transcriptional activity (e.g., using a reporter gene), enzymatic activity (e.g., via a property of the substrate), activity of second messengers, immunogenicty and changes in cellular morphology or other cellular characteristics. Appropriate screening assays may use a wide range of detection methods including fluorescent, radioactive, colorimetric, spectrophotometric, and amperometric methods, to provide a read-out for the particular molecular event detected.

Preferred screening assays are high throughput or ultra high throughput and thus provide automated, cost-effective means of screening compound libraries for lead compounds (Fernandes P B, 1998, supra; Sundberg S A, Curr Opin Biotechnol 2000, 11:47-53). Many high throughput screening assays utilize fluorescence technologies, including fluorescence polarization, time-resolved fluorescence, and fluorescence resonance energy transfer. These systems offer means to monitor protein-protein or DNA-protein interactions in which the intensity of the signal emitted from dye-labeled molecules depends upon their interactions with partner molecules (e.g., Selvin P R, Nat Struct Biol (2000) 7:730-4; Fernandes P B, Curr Opin Chem Biol (1998) 2:597-603; Hertzberg R P and Pope A J, Curr Opin Chem Biol (2000) 4:445-451). Other assays include radioactive, colorimetric, spectrophotometric, and amperometric detection methods.

Assays for these preferred classes of protein targets have been reported in the literature and are well known to those skilled in the art. Examples of assays that may be used to test the activity of the preferred targets are described below.

Protein kinases catalyze the transfer of gamma phosphate from adenosine triphosphate (ATP) to a serine, threonine or tyrosine residue in a protein substrate. Radioassays, which monitor the transfer from [gamma-³²P or -³³P]ATP, are frequently used to assay kinase activity. Separation of the phospho-labeled product from the remaining radio-labeled ATP can be accomplished by various methods including SDS-polyacrylamide gel electrophoresis, filtration using glass fiber filters or other matrices which bind peptides or proteins, and adsorption/binding of peptide or protein substrates to solid-phase matrices allowing removal of remaining radiolabeled ATP by washing. In one example, a scintillation assay monitors the transfer of the gamma phosphate from [gamma-³³P] ATP to a biotinylated peptide substrate. The substrate is captured on a streptavidin coated bead that transmits the signal (Beveridge M et al., J Biomol Screen (2000) 5:205-212). This assay uses the scintillation proximity assay (SPA), in which only radio-ligand bound to receptors tethered to the surface of an SPA bead are detected by the scintillant immobilized within it, allowing binding to be measured without separation of bound from free ligand. Other assays for protein kinase activity may use antibodies that specifically recognize phosphorylated substrates. For instance, the kinase receptor activation (KIRA) assay measures receptor tyrosine kinase activity by ligand stimulating the intact receptor in cultured cells, then capturing solubilized receptor with specific antibodies and quantifying phosphorylation via phosphotyrosine ELISA (Sadick M D, Dev Biol Stand (1999) 97:121-133). Another example of antibody based assays for protein kinase activity is TRF (time-resolved fluorometry). This method utilizes europium chelate-labeled anti-phosphotyrosine antibodies to detect phosphate transfer to a polymeric substrate coated onto microtiter plate wells. The amount of phosphorylation is then detected using time-resolved, dissociation-enhanced fluorescence (Braunwalder A F, et al., Anal Biochem Jul. 1 1996;238(2): 159-64). Generic assays may be established for protein kinases that rely upon the phosphorylation of substrates such as myelein basic protein, casein, histone, or synthetic peptides such as polyGlutamate/Tyrosine and radiolabeled ATP.

Phosphoinositide kinases catalyze the phosphorylation of phosphatidylinositol substrates. Assays for lipid kinase activity may use labeled, such as radio-labeled substrates to detect transfer of a phosphate to lipid substrates. In one example, assays may use chromatography techniques to detect phosphorylation (Sbrissa D et al., 1999, J Biol Chem 274:21589-21597). In another example, an assay uses “FlashPlate” technology (U.S. Pat. No. 5,972,595), in which the hydrophobic substrate is immobilized on a solid support in each well of a multi-well plate. Phosphorylation of the substrate with a radio-labeled phosphate is measured as an increase in bound radioactivity, which is detected by the close proximity of the scintillant.

Protein phosphatase catalyze the removal of a gamma phosphate from a serine, threonine or tyrosine residue in a protein substrate. Since phosphatases act in opposition to kinases, appropriate assays measure the same parameters as kinase assays. In one example, the dephosphorylation of a fluorescendy labeled peptide substrate allows trypsin cleavage of the substrate, which in turns renders the cleaved substrate significantly more fluorescent (Nishikata M et al., Biochem J (1999) 343:35-391). In another example, fluorescence polarization monitors direct binding of the phosphatase with the target; increasing concentrations of phosphatase increases the rate of dephosphorylation, leading to a change in polarization (Parker G J et al., (2000) J Biomol Screen 5:77-88).

Assays for inositol phosphatase activity may use labeled, such as fluorescently labeled or radio-labeled substrates to detect removal of a phosphate from a phosphatidylinositol substrate. In one example, an assay uses “FlashPlate” technology, and dephosphorylation of the substrate is measured as a decrease in bound radioactivity, which is detected by the close proximity of the scintillant. Other assays for detecting phosphoinositide phosphatase activity are known in the art (see, e.g., U.S. Pat. Nos. 6,001,354 and 6,238,903).

Proteases are enzymes that cleave protein substrates at specific sites. Exemplary assays detect the spectral properties of an artificial substrate, which are altered by protease-mediated cleavage. In one example, synthetic protease (caspase) substrates contain four amino acid proteolysis recognition sequences, separating two different fluorescent tags; fluorescence resonance energy transfer detects the proximity of these fluorophores, which indicates whether the substrate is cleaved (Mahajan N P et al., Chem Biol (1999) 6:401-409).

Endogenous protease inhibitors may inhibit protease activity. In an example of an assay developed for either proteases or protease inhibitors, a biotinylated substrate is coated on a titer plate and hydrolyzed with the protease; the unhydrolyzed substrate is quantified by reaction with alkaline phosphatase-streptavidin complex and detection of the reaction product. The activity of protease inhibitors correlates with the activity of the alkaline phosophatase indicator enzyme (Gan Z et al., Anal Biochem 1999) 268:151-156).

DNA topoisomerases are multifunctional nuclear enzymes that help maintain proper DNA topology. Appropriate assays may measure topoisomerases' catalytic cleavage and/or re-ligation activity, which generally involve the formation of covalent bond between a tyrosine residue and the DNA substrate. For instance, an assay for Vaccinia topoisomerase ligation activity uses a synthetic oligonucleotide substrate with an attached 3′ tyrosine analog whose release during ligation is measured spectrophotometrically (e.g., Woodfield et al., Nucleic Acids Research (2000) 28:3323-3331).

Helicases are involved in unwinding double stranded DNA and RNA. In one example, an assay for DNA helicase activity detects the displacement of a radio-labeled oligonucleotide from single stranded DNA upon initiation of unwinding (Sivaraja M et al., Anal Biochem (1998) 265:22-27). An assay for RNA helicase activity uses the scintillation proximity (SPA) assay to detect the displacement of a radio-labeled oligonucleotide from single stranded RNA (Kyono K et al., Anal Biochem (1998) 257: 120-126).

Polymerases catalyze the extension of newly synthesized DNA or RNA chains. Their activity may be monitored in an assay that uses labeled nucleotide analogs. For instance, a colorimetric polymerase assay monitors RNA synthesis using labeled ATP and GTP (Vassiliou W et al., Virology (2000) 274:429-437).

Phosphodiesterases (PDEs) catalyze the cleavage of phosophodiester bonds and thereby degrade cyclic nucleotides, such as cyclic AMP, which regulate many cellular processes. The activity of cyclic nucleotide PDEs may be tested using the selective precipitation of a labeled 5′ nucleotide product (Schilling R J et al., Anal Biochem (1994) 216: 154-158). Alternatively the scintillation proximity assay may be used to detect the radio-labeled 5′ nucleotide product (Bardelle C et al., Anal Biochem (1999) 275:148-155).

Phospholipases hydrolyze phosopholipds into lipid products that may serve as second messengers or may be precursors in the biosynthesis of various biologically active products. In one example, a spectrophotometric assay detects phospholipase A activity by measuring the absorbance of a reaction product produced after hydrolysis of a synthetic phospholipid analogue, carbonothioate phospholipid (Yu L et al., Anal Biochem (1998) 265:35-41).

Peptidyl-prolyl isomerase (PPLase) proteins, which include cyclophilins, FK506 binding proteins and paravulins, catalyze the isomerization of cis-trans proline peptide bonds in oligopeptides and are thought to be essential for protein folding during protein synthesis in the cell. Spectrophotometric assays for PPIase activity can detect isomerization of labeled peptide substrates, either by direct measurement of isomer-specific absorbance, or by coupling isomerization to isomer-specific cleavage by chymotrypsin (Scholz C et al., FEBS Lett (1997) 414:69-73; Janowski B et al., Anal Biochem (1997) 252:299-307; Kullertz G et al., Clin Chem (1998) 44:502-8). Alternative assays use the scintillation proximity or fluorescence polarization assay to screen for ligands of specific PPIases (Graziani F et al., J Biolmol Screen (1999) 4:3-7; Dubowchik G M et al., Bioorg Med Chem Lett (2000) 10:559-562).

Many different metabolic enzymes are amenable to assay development, due to well-characterized substrates and active sites, generally simple reaction mechanisms, and a general conservation in reaction mechanism. Numerous assays for metabolic enzymes have been developed. For instance, fatty acid desaturases and glycosyltransferases are two classes of metabolic enzymes that have been contemplated for drug targets and are similarly appropriate as pesticide targets. Fatty acid desaturases catalyze the insertion of double bonds into saturated fatty acid molecules. In one embodiment, radioassays for inhibitors of delta-5 and delta-6 fatty acid desaturase activity use thin layer chromatography to detect conversion of fatty acid substrates (Obukowicz et al., Biochem Pharmacol (1998) 55:1045-1058). Glycosyltransferases mediate changes in glycosylation patterns that, in turn, may affect the function of glycoproteins and/or glycolipids and, further downstream, processes of development, differentiation, transformation and cell-cell recognition. An assay for glycosyltransferase uses scintillation methods to measure the transfer of carbohydrate from radiolabeled sugar-nuecleotide donor to a synthetic glycopolymer acceptor that is coupled to polyacrylamide and coated on plastic microtiter plates (Donovan R S et al., Glycoconj J (1999) 16:607-615).

Ion channels mediate essential physiological functions, including fluid secretion, electrolyte balance, bioenergetics, and membrane excitability. Assays for channel activity can incorporate ion-sensitive dyes or proteins or voltage-sensitive dyes or proteins, as reviewed in Gonzalez J E et al. (Drug Discovery Today (1999) 4:431-439). Alternative methods measure the displacement of known ligands, which may be radio-labeled or fluorescently labeled (e.g., Schmid E L et al., Anal Chem (1998) 70:1331-1338).

The nuclear hormone receptor super-family comprises ligand-activated transcription factors that allow a cell to respond to changes in the extracellular environment. These receptors have well-characterized, modular domains, facilitating design of inhibitory compounds. Their activity can be monitored through assays that depend on ligand-dependent interaction with co-activators or a transcriptional readout (Nishikawa J et al., Toxicol Appl Pharmacol (1999) 154:76-83; Burris T P et al., Mol Endocrinol (1999) 13:410-7). Additionally, currently available crystal structures for nuclear hormone receptors can be used for the rational design of antagonists (Shapira M et al., Proc Natl Acad Sci U S A (2000) 97:1008-1013).

G-protein-coupled receptors (GPCRs) comprise a large family of cell surface receptors that mediate a diverse array of biological functions. They selectively respond to a wide variety of extracellular chemical stimuli to activate specific signaling cascades. Assays may measure reporter gene activity or changes in intracellular calcium ions, or other second messengers (Durocher Y et al., Anal Biochem (2000) 284: 316-326; Miller T R et al., J Biomol Screen (1999) 4:249-258). Such assays may utilize chimeric G-alpha proteins that will couple to many different GPCRs and thus facilitate “universal” screening assays (Coward P et al., Anal Biochem (1999) 270:242-248; Milligan G and Rees S et al., Trends Pharmacol Sci (1999) 20:118-124).

GPCRs exert their effects through heterotrimeric G proteins, which cycle between active GTP- and inactive GDP-bound forms. Receptors catalyze the activation of G proteins by promoting exchange of GDP for GTP, while G proteins catalyze their own deactivation through their intrinsic GTPase activity. GEFs accelerate GDP dissociation and GTP binding, while GAPs stimulate GTP hydrolysis to GDP. The same assays used to monitor GPCR activity may thus be applied to monitor the activity of GEFs or GAPs. Alternatively, GEF activity may be assayed by the release of labeled GDP from the appropriate GTPase or by the uptake of labelled GTP; GAP activity may be monitored via a GTP hydrolysis assay using labeled GTP (e.g., Jones S et al., Molec Biol Cell (1998) 9:2819-2837).

Sometimes, the establishment of the appropriate assay will depend on the identification of appropriate protein binding partners for the given target. Yeast two-hybrid and variant screens offer preferred, established methods for determining protein-protein interactions (reviewed in, e.g., Fashema S F et al., Gene (2000) 250:1-14; Drees B L Curr Opin Chem Biol (1999) 3:64-70; Vidal M and Legrain P Nucleic Acids Res (1999) 27:919-29). Mass spectrometry offers alternative preferred methods for the elucidation of protein complexes (reviewed in, e.g., Pandley A and Mann M, Nature (2000) 405:837-846; Yates J R 3^{rd}, Trends Genet (2000) 16:5-8)

Even when a particular target may not be associated with an existent high-throughput assay, it still may be possible to monitor its activity using high-throughput cell-based or cell-free methods. Since noncovalently associated multi-protein complexes mediate many biological processes, many effective chemical antagonists will function by disrupting such complexes. If the putative target belongs to a complex that is essential for protein function, appropriate assays may monitor complex formation or survival, instead of function per se. A variety of assays are available to detect the activity of proteins that have specific binding activity. Exemplary assays use fluorescence polarization, fluorescence polarization, and laser scanning techniques to measure binding of fluorescently labeled proteins, peptides, or other molecules (Lynch B A et al., 1999, Anal Biochem 275:62-73; Li H Y, 2001, J Cell Biochem 80:293-303; Zuck P et al., Proc Natl Acad Sci USA 1999, 96: 11122-11127). In another example, binding activity is detected using the scintillation proximity assay (SPA), which uses a biotinylated peptide probe captured on a streptavidin coated SPA bead and a radio-labeled partner molecule. The assay specifically detects the radio-labeled protein bound to the peptide probe via scintillant immobilized within the SPA bead (Sonatore L M et al., 1996, Anal Biochem 240:289-297). An appropriate cell-based assay is based on protein complementation, in which two proteins in a complex are fused to complementary fragments of the enzyme dihydrofolate reductase (DHPR). Enzyme activity of DHFR depends on proper folding of the two fragments, which in turn depends upon binding of the two complexed proteins. Two properties of DHFR can be assayed. DHFR confers viability to DHFR-negative cells in a cell survival assay, or DHFR binds a fluorescent substrate in a reporter assay (Remy I and Michnick S W, Proc Natl Acad Sci (1999) 96:5394-5399).

Cell-based screening assays usually require systems for recombinant expression of the target protein and any auxiliary proteins demanded by the particular assay. Cell-free assays often use recombinantly produced purified or substantially purified proteins. Appropriate methods for generating recombinant proteins produce sufficient quantities of proteins that retain their relevant biological activities and are of sufficient purity to optimize activity and assure assay reproducibility. Techniques for the expression, production, and purification of proteins are well known in the art (e.g., Higgins S J and Hames B D (eds.) Protein Expression: A Practical Approach, Oxford University Press Inc., New York 1999; Stanbury P F et al., Principles of Fermentation Technology, 2^{nd} edition, Elsevier Science, New York, 1995; Doonan S (ed.) Protein Purification Protocols, Humana Press, New Jersey, 1996; Coligan J E et al, Current Protocols in Protein Science (eds.), 1999, John Wiley & Sons, New York). Assays for protein targets may employ any suitable methods for expression, production, and purification of required proteins. These methods include means to quantify and verify the activity of expressed or purified proteins. Once a protein is obtained, it is generally quantified and its activity measured by appropriate methods, such as immunoassay, bioassay, or other measurements of physical properties, such as crystallography.

Pesticide Development

Pesticidal compounds include chemical agents, referred to in the art as “small molecule” compounds, which are typically organic, non-peptide molecules, having a molecular weight less than 10,000, preferably less than 5,000, more preferably less than 1,000, and most preferably less than 500. This class of modulators includes chemically synthesized molecules, for instance, compounds from combinatorial chemical libraries. Synthetic compounds may be rationally designed or identified based on known or inferred properties of the target protein or may be identified by screening compound libraries. Compound classes such as the organophosphates, pyrethroids, carbamates, and organochlorines typify this type of small molecule pesticide. Alternative pesticides include natural products, particularly secondary metabolites from organisms such as plants or fungi, which can also be identified by screening compound libraries for target-modulating activity. Methods for generating and obtaining compounds are well known in the art (Schreiber S L, Science (2000) 151: 1964-1969; Radmann J and Gunther J, Science (2000) 151:1947-1948). Other pesticides include proteinaceous toxins such as the Bacillus thuringiensis Crytoxins (Gill et al., Annu Rev Entomol (1992) 37:615636) and Photorabdus luminescens toxins (Bowden et al., Science (1998) 280:2129-2132); and nucleic acids such as dsRNA or antisense nucleic acids that interfere with target gene activity.

Pesticides can be delivered by a variety of means including direct application to pests or to their food source. Additionally, toxic proteins and pesticidal nucleic acids can be administered using biopesticidal methods, for example, by viral infection or by transgenic plants that have been engineered to produce interfering nucleic acid sequences or encode the toxic protein, which are ingested by plant-eating pests.

For any candidate pesticidal compound, further testing is done to determine efficacy against the target organism, toxicology, side effects on non-target organisms, environmental impact, etc.

### EXAMPLES

### Example 1

### Drosophila Screen for Pesticide Targets

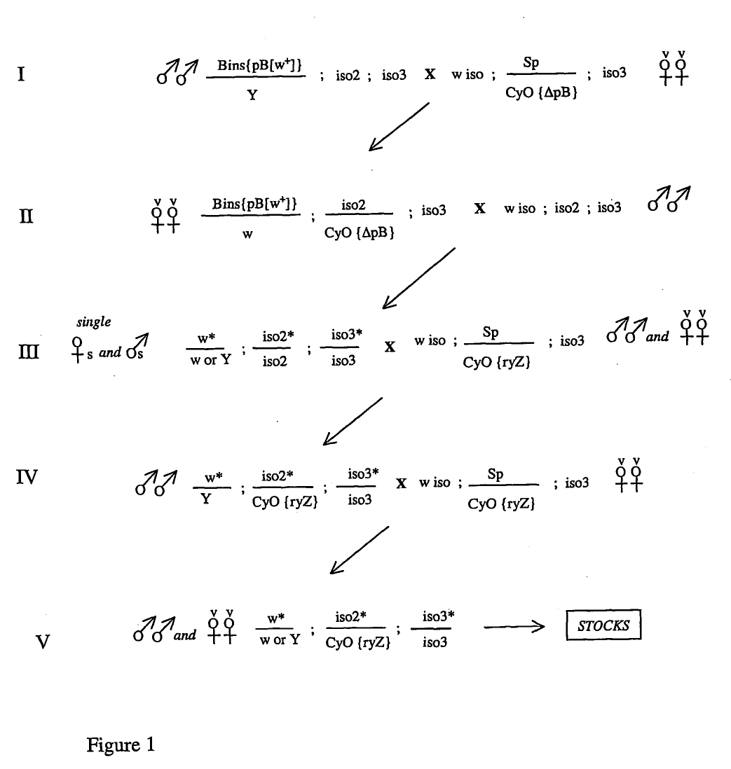

FIG. 1 depicts the genetic crosses for conducting a screen for pesticide targets in Drosophila. The markers and the conventions used to diagram genetic crosses are well known to those skilled in the art and are further described in Lindsley D L and Zimm G G (The Genome of Drosophila melanogaster (1992) Academic Press). An ˜5 kb piggyBac transposon (“pB[w⁺]”), which contains the white (w⁺) minigene (http://flybase.bio.indiana.edu/.bin/tpseq.html?FBms0000515), flanked by direct FRT sites (GI172190, nt 676-723) is used as the ammunition vector and is introduced by standard germline transformation techniques (Ashburner, supra) into an isogenic w⁻ background with the X-balancer chromosome Binsnscy (“Bins,” http://flybase.bio.indiana.edu/.bin/fbidq.html?FBab0010488). In order to be able to distinguish novel insertions from the parental insertion, parental hosts in which the piggyBac transposon had inserted on Bins are selected. The term “iso” refers to an isogenized chromosome.

A first genetic cross (I) brought together the parental piggyBac ammunition vector and the source of piggyBac transposase under the control of the Drosophila alpha1-tubulin 5′ UTR and K10 3′ UTR (abeled “ΔpB” in FIG. 1), which had been transferred to the CyO second chromosome balancer, as described above. The fly stock providing the transposase was also in a w⁻ isogenic background and carried the dominant Sp second chromosome marker in trans to CyO. Dysgenic female progeny that harbored both parental ammunition vector and transposase were recovered. They were distinguished based on mottled eye color, indicating mobilization of the ammunition vector in these animals' somatic tissue, and as Bar⁻ and Sp⁺ or Cy⁻. Dysgenic progeny were out-crossed (II) to isogenic, w⁻ flies to segregate away the transposase and recover the novel insertions. From the second cross, male and female progeny with novel insertions (indicated by “*;” progeny may have novel insertions on any chromosome) were identified by w⁺ eye color that segregated away from the marked Bins balancer. These animals were singly mated (III) to animals with a dominant second chromosome marker Sp in trans to a lacZ-tagged CyO balancer (“CyO {ryZ}”), in order to recover males representing each novel insertion. These males were re-crossed (IV) to females containing the dominant second chromosome marker Sp in trans to the lacZ-tagged CyO balancer. Standard genetic methods based on phenotypes of progeny from the fourth cross were used to determine the chromosomal position of each insertion. Animals with insertions on the X chromosome yielded exclusively w⁻ sons and exclusively w⁺ daughters. Animals with insertions on the 2^{nd} chromosome yielded progeny such that all Sp⁻ progeny were either w⁺ or Cy⁻, and all Cy⁻ progeny were either w⁺ or Sp⁻. If neither situation applied, insertions were on the third chromosome, or a negligible percentage on the fourth chromosome. Sibling males and females that harbored the novel insertions were collected and crossed (V) to found a stock. Progeny of the sibling cross were scored, based on eye color, for the presence or absence of homozygous animals. Stocks that yielded progeny of a uniform eye color were scored as homozygous lethal, whereas stocks that yielded some animals with darker eye color than siblings and parents were homozygous viable. Stocks with second chromosome insertions could additionally be scored for absence of the non-balancer class, indicating that the insertion was homozygous lethal.

These methods were used to generate a collection of approximately 5600 stable insertion lines. Approximately 1350 of these had lethal insertions.

### Example 2

### Moth Screen for Pesticide Targets

A screen for pesticide targets in the tobacco budworm, Heliothis virescens, uses gene expression analysis to identify the new insertions and prioritize those likely to be essential for viability. The screen uses an enhancer trap system with GAL4/UAS components, as well as three fluorescent proteins with distinct emission spectra (Tsien R, Annu. Rev Biochem (1998) 67:509-544) to mark the various transgenic components. In addition to the ammunition vector, two other transposable elements are used to introduce the transposase source and the UAS-dependent reporter gene.

The ammunition vector (“YFP-GAL4”) carries GAL4 under a minimal promoter and is marked with yellow fluorescent protein (YFP) under the control of a strong promoter. YFP serves as the primary marker, which is used to detect the transformation of the initial host animals, and to mark all subsequent progeny that carry the transgene. The GAL4 transgene relies on insertion site-dependent regulatory elements for its expression. A second transgene carries a GFP reporter under UAS control (“GFP-UAS”), such that in animals containing both GFP-UAS and YFP-GAL4, GFP is expressed in a pattern that reflects the regulatory elements of the gene into which the ammunition vector inserts. GFP is additionally under control of the 3×P3 synthetic promoter (Berghammer, supra), which directs expression in the eye in all animals bearing the transgene. Finally, the third transgene carries a source of transposase and is marked with a CPP marker gene (“CFP-tpase”).

The transgenes are initially transferred to separate host animals by injection. Appropriate parental hosts are obtained by standard procedures for crossing injected animals to wild-type animals and selecting progeny that display the appropriate marker gene to found the stocks. The YFP-GAIA and GFP-UAS stocks require additional analysis prior to selecting suitable stocks for the screen. Since the UAS regulatory sequences depend on the presence of the GAL4 transcriptional activator to promote expression of linked genes, suitable parental GFP-UAS stocks display no GFP fluorescence except for the 3×P3-linked GFP expression in eye tissue. Individuals from the YFP-GAL4 transformant stocks are crossed to individuals carrying the GFP-UAS transgene, and progeny are analyzed for GFP expression, which reflects endogenous regulatory information proximal to the YPP-GAL4 insertion site. Preferred YFP-GAL4 stocks confer consistent expression in restricted pattern. Alternatively, a stock that confers no or low-level expression, reflecting an absence of nearby trans-acting regulatory sequences, may be selected if at least one of the YFP-GAL4 stocks confers strong, consistent expression, confirming the ability of the GFP-UAS transgene to be activated via GAL4.

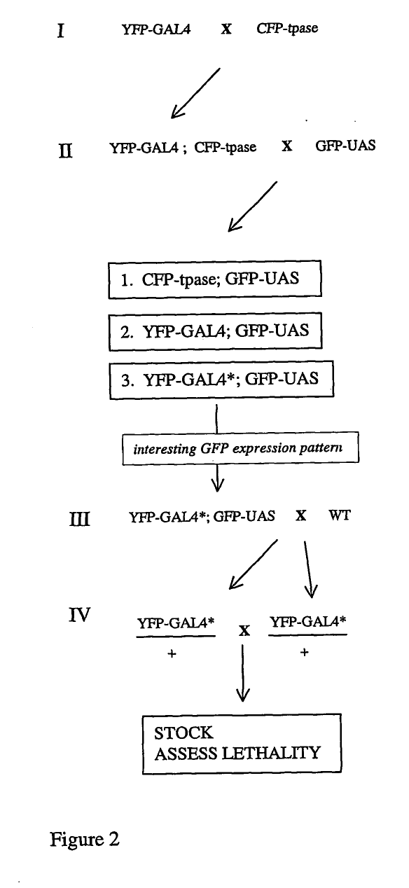

Once the requisite transgenic stocks are created and selected, genetic crosses bring together the various elements. FIG. 2 depicts the genetic crosses used to conduct the screen; the conventions used to diagram genetic crosses are well known to those skilled in the art. A first genetic cross (I) brings together YFP-GAL4 and CFP-tpase, in order to mobilize the ammunition vector. Dysgenic progeny, which display both YFP and CFP markers, are crossed (II) to animals carrying GFP-UAS. From this cross, three classes of animals (1, 2, and 3) are obtained. Animals that express GFP in a novel expression pattern (3), and therefore contain a new insertion of the ammunition vector, are selected. The “*” indicates a new insertion. Animals that express GFP in the parental expression pattern (2) are not selected, nor are animals that display the CFP marker (1) and therefore carry the transposase. Preferred progeny display broad expression of the GFP-UAS reporter gene and/or display expression in essential tissues, such the gut, the nervous system, and musculature. Gene expression in these tissues is often indicative of essential function. Preferred progeny also lack the transposase source, as evidenced by absence of the CFP marker. Individual animals containing YFP-GAL4 and GFP-UAS are out-crossed (III) to wild-type (WT) animals in order to founder stocks. Siblings of this outcross that are heterozygous for the ammunition vector are crossed to each other (IV) to assess whether the new insertions are lethal. Lethality is assessed according to principles of Mendelian segregation. Preferably at least 25, more preferably at least 50 progeny are available for analysis. If the insertion is homozygous viable, three-quarters of progeny should express the YFP marker. If it is homozygous lethal, only two-thirds should express the marker. If the insertion is not lethal, progeny can further be observed for defects in fecundity, locomotion, and eating habits, etc. If such defects occur only in animals that display the YFP marker and occur in one quarter (or fewer) animals, it is likely that the ammunition vector insertion is responsible.

### Example 3

### Evidence of Splice Trapping in a Collection of Drosophila Insertions

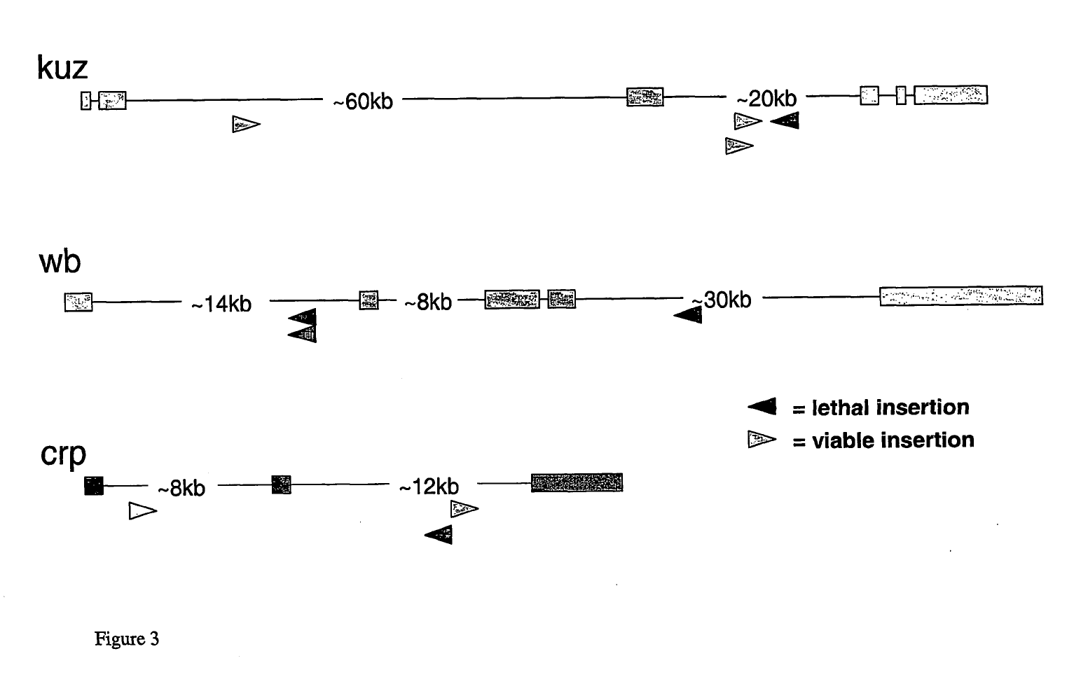

A collection of isogenic Drosophila lines with piggyBac insertions was generated. The piggyBac transposon contained a single splice trap cassette, namely, the white minigene (mini-w⁺), which has previously been shown to promote aberrant mis-splicing from a P-element vector (Goodwin, supra). Insertions in three genes, kuzbanian (kuz; http://flybase2.bio.indiana.edu/.bin/fbidq.html?FBgn0015954), wing blister (wb; http://flybase2.bio.indiana.edu/.bin/fbidq.html?FBgn0004002), and cropped (crp; http://flybase.bio.indiana.edu/.bin/fbidq.htrnl?FBgn0001994) were analyzed in order to correlate the position of the splice trap sequences to a lethal phenotype. In order for the splice trap sequences to be effective, the direction of the white minigene (mini-w⁺) should be in the same relative orientation as the endogenous gene with the piggyBac insertion (i.e., the direction of transcription should be the same). FIG. 3 depicts the results of the analysis. There were four insertions in kuz. One in the second intron and two in the 3^{rd} intron were inserted such that mini-w⁺ was in the opposite orientation as kuz; these insertions were not lethal. A fourth insertion in the 3^{rd} intron inserted such that mini-w⁺ was in the same orientation as kuz, and this insertion was lethal. There were three insertions in wb, two in the 1^{st} intron and one in the 4^{th}. All were inserted such that mini-w⁺ was in the same orientation as wb, and all insertions were lethal. There were three insertions in crp. One in the 1^{st} intron and one in the 2^{nd} intron were inserted such the mini-w⁺ was in the opposite orientation as crp; these were not lethal. The third, in the 2^{nd} intron, was inserted with mini-w⁺ in the same orientation as crp, and this was lethal.

Thus, data from kuz, wb, and crp correlate lethality to orientation of the transposon and is completely consistent with the hypothesis that aberrant splicing into the mini-w⁺ cassette is responsible for lethality. These data furthermore show that the method of using a splice trap transposon with a single splice trap cassette and correlating lethality to orientation of the transposon is an effective means to assess the effectiveness of new splice trap sequences.

### Example 4

### Insertion and Mobilization Characteristics of piggyBac and XP Transposons

Transformation and Remobilization

The piggyBac vector pB[w⁺] was constructed by cloning the Drosophila white (w⁺) gene into the HpaI site (GTTAAC) within a complete piggyBac transposon (SEQ ID NO:1). pB[w⁺] was used in the first phase of screening as we assessed the utility of piggyBac as a forward mutagen. Subsequent phases of the screen also utilized a piggyBac transposon marked with w but with additional functionalities built in such as FRT recombination sites, Su(Hw) sequences, and a UAS site.

Our initial piggyBac transformation experiments utilized pB[w⁺] and a heat shock helper plasmid, a stable source of transposase constructed by cloning the piggyBac coding sequence under control of the Drosophila Hsp70 promoter into a P element and mobilizing it onto the CyO balancer chromosome. We recovered primary transformants at an average rate of 7% (in the absence of heat shock). Primary inserts from five of the lines were subsequently remobilized with frequencies of new insertion ranging from 2 to 15%. Although the heat shock promoter can allow a basal level of expression we recovered no secondary remobilizations in the absence of heat shock. We tested a range of induction times and found the best conditions were daily 1 hour heat shocks in a 37° C. water bath from days 3-10 of development. Still, remobilizations were recovered at a relatively low frequency (10%). With the aim of improving the rate of mobilization we engineered the transposase source, pP[a-tub:pBac], as described above, to contain a constitutive promoter and a germline-stable 3′ UTR; P[a-tub:pBac] was integrated onto CyO. By also selecting among the lines for an easily mobilized ammunition transposon, which was selected among inserts hopped onto the Binsnscy balancer chromosome, we were able to obtain a much improved rate (60%) in the large scale genetic screen, in which new insertions were collected from dysgenic females. XP remobilization events were collected in a similar manner utilizing the standard chromosomal source of transposase, delta 2-3 at 99B XP elements were found to hop at a similar frequency (50%). Expression of the w⁺ transgene among all lines ranged from very pale yellow to dark red.

Characterization of Inserts

Transposon insertions (inserts) were assigned to a chromosome by standard mapping crosses; multiple inserts were segregated from each other when possible. Flanking genomic sequence from 5′ and/or 3′ ends was obtained by inverse PCR (iPCR) and was used in BLAST comparison to the fly genome. We wrote software to automate the recovery and handling of sequence data within a laboratory information management system (LIMS). Flanking sequence was first masked of any vector, trimmed for quality, and searched against Drosophila genomic sequence deposited in GenBank. The completion of the Drosophila Genome Project allowed us to associate flanking sequence with a unique genomic region for 76% of piggyBac lines and 64% of XP lines. The larger percentage of piggyBac inserts with unique locations is explained by both a greater success rate for piggyBac iPCR and recovery of longer flanking sequences. Lines from which flanking sequence could not be recovered by iPCR, was of insufficient length to determine its unique position, or mapped to multiple locations were excluded from the analysis.

We found a wide distribution of inserts across the X and two major autosomes. Insertion hotspots are apparent for both transposons. Defining a hotspot as a 50 kb interval containing 30 or more insertions, we found 21 P (XP) hotspots, many of which correspond to those previously identified. We also found 8 piggyBac hotspots. However, two of the piggyBac hotspots (on X and III) probably result from local transpositions of ammunition elements. Excluding local hops as true hotspots, we found fewer than one third as many piggyBac hotspots from 1.6 times as many insertions. Thus, piggyBac distributes itself considerably more randomly than P.

We examined the piggyBac insertion spectrum by determining the relative location of insertions among those associated with genes found in FlyBase (www.flybase.bio.indiana.edu). Compared with P, piggyBac was significantly less likely to insert in 5′ sequences. Insertions were recovered in 5′ UTR, introns, exons, as well as 3′ UTR. A bias may exist for intronic insertion. This is not surprising given the known high AT content of Drosophila introns and piggyBac's tetranucleotide target site (TTAA). Compared to P, the greater likelihood of piggyBac to insert between start and stop codons may help to explain the larger percentage of lethal inserts recovered. Typical P screens recover 10-15% of insertions in genes required for viability or fertility (Cooley L et al., Science (1988) 239:1121-8.). We recovered recessive lethals at a rate of 22% among all piggyBac lines and 17% of XPs.

Secondary Hits and Excision Analysis

We assessed the possibility of hit and run events for piggyBac at both gross and fine scales. At the gross level, 99% of recessive lethal phenotypes recovered from the piggyBac screens mapped to the same chromosome as the transposon. In addition approximately all piggyBac lethal insertions tested failed to complement deficiency-bearing chromosomes that were known to delete the mutated locus. Thus, if second site lethal mutations were present they were likely local. We subjected several of these lines to an additional test, generating heritable (germline) excisions and analyzing their progeny both genetically and molecularly.

We found that each piggyBac excision reverted the lethal phenotype of the chromosome, indicating the absence of a background lethal such as a hit and run event. Each excision also complemented a corresponding deficiency chromosome, further supporting the “precise” nature of the events in a genetic sense. Using primers flanking the insertion site, the sequence of the excision chromosome around the original insertion site was determined. In all cases, the TTAA target site duplication was repaired to wild-type. Results are shown in Table 1. The “Non-Complementing Deficiencies” are given as Bloomington stock collection numbers.

TABLE 1

| | | Non- | | | Excisions | Precise |
|---|---|---|---|---|---|---|
| Lethal | | Complementing | Independent | Viable | Complementing | Molecular |
| Stock | Chromosome | Deficiency | Excisions | Excisions | Deficiency | Excision |
| 146 | 2 | B-384 | 10 | 10/10 | 10/10 | 10/10 |
| 80 | 2 | B-3084 | 10 | 10/10 | 10/10 | 10/10 |
| 221 | 2 | B-1006 | 10 | 10/10 | 10/10 | 10/10 |
| 93 | 2 | B-442 | 10 | 10/10 | 10/10 | 10/10 |
| 119 | 3 | B-1910 | 10 | 10/10 | 10/10 | 10/10 |
| 83 | 3 | B-3011 | 5 | 5/5 | 5/5 | 5/5 |

Gene Saturation

An ideal transposon collection would contain at least one tag for every gene. The ongoing public P-element Gene Disruption Project (Spradling A C et al., Genetics (1999) 153:135) has the goal of disrupting all Drosophila genes regardless of phenotype. Randomly generated inserts can be expected to yield the greatest proportion of novel gene tags early in a screen. Diminishing returns then means that tagging all remaining genes requires a disproportionately large effort. The large number of lines from our XP and piggyBac screens allowed us to examine the gene tagging frequency of both elements. To measure saturation we first defined a gene as tagged if the transposon inserted between the start and stop codons or was within 1,000 bp of the start codon (presuming disruption of promoter/regulatory elements). Genes were defined according the curation of the BDGP Drosophila Gene Collection (DGC r1.0), which represents a quality controlled set of 5,849 full-length cDNAs. The gene dense nature of the Drosophila genome results in some transposons being counted as tagging more than one gene. As expected due to hotspots, the rate of return of new genes tagged by XP declined sharply. Conversely, piggyBac continued to yield new gene hits at a reasonable frequency even after 6,000 insertions. It has been estimated that with the use of P alone 87% genome saturation might require screening up to 150,000 insertions. The hotspot phenomenon implies that use of P alone to attempt to tag all Drosophila genes will necessarily generate thousands of redundant lines that first must be analyzed and then discarded. The more random distribution of piggyBac elements favors its choice for saturation tagging.

Element Stability

We have seen no evidence of cross-mobilization—neither by piggyBac transposase nor of piggyBac transposons—in our Drosophila stocks. The stock containing a constitutively active piggyBac transposase has been quite stable for over year. Early piggyBac insertions from primary transformation have been maintained stably for over two years. We have found no detectable interactions between piggyBac transposase and P element ends (and vice versa). Indeed, we introduced piggyBac transposase into the Drosophila genome by P transformation. A stock with a piggyBac element containing delta 2-3 P transposase likewise has been maintained stably. The lack of cross-mobility between the two elements makes it easy to contemplate a piggyBac screen in a P background (and vice versa). Southern analysis with a piggyBac probe did not detect related sequences in our Drosophila stocks.

1) A method of identifying a pesticidal agent comprising:
a) generating a transposon insertion in an insect cell within a host system;
b) determining if the transposon insertion is a lethal insertion;
c) identifying alethal gene corresponding to the lethal insertion identified in (b);
d) screening candidate pesticidal agents for ability to specifically inhibit function of a protein product of the lethal gene identified in (c), wherein an agent that inhibits function of the protein product is identified as a pesticidal agent.

2) The method of claim 1 wherein the insect cell is an in vivo germline cell, and said host system is an insect.

3) The method of claim 6 wherein the transposon insertion is generated by steps comprising:
(i) obtaining an insect that has been genetically modified by insertion of a transposon into a first site in the genome of said gernline cell;
(ii) introducing a construct comprising a transposase into said germline cell; and
(iii) causing expression of the transposase which mediates integration of the transposon into a second site in the genome of the germline cell.

4) The method of claim 1 wherein the transposon is an XP element.

5) The method of claim 1 wherein the transposon is piggyBac.

6) The method of claim 4 or 5 wherein said transposon comprises splice trap sequences.

7) The method of claim 1 wherein the insect cells are from Drosophila.

8) The method of claim 1 wherein the insect cells are from a crop pest species.

9) The method of claim 1 wherein the lethal gene encodes an enzyme or soluble protein.

10) The method of claim 9 wherein the enzyme or soluble protein is selected from the group consisting of protein kinase, protein phosphatase, protease, protease inhibitor, topoisomerase, helicase, polymerase, phosphodiesterase, phospholipase, prolylisomerase, nuclear hormone receptors, GTPase activating protein (GAP), and guanine nucleotide exchange factors (GEF), and a metabolic enzyme.

11) The method of claim 1 wherein the lethal gene encodes a membrane protein.

12) The method of claim 11 wherein the membrane protein is selected from the group consisting of G protein coupled receptor (GPCR), protein kinase receptor, ligand-gated ion channel, voltage dependent ion channel, and transporter.

13) A biological array comprising transgenic insect lines, wherein each line is randomly mutated with at least one piggyBac transposon, and wherein essentially every gene in the insect's genome is mutated by a piggyBac transposon in at least one insect line.

14) A biological array comprising transgenic Drosophila lines, each line randomly mutated with at least one piggybac transposon or XP transposon, and wherein essentially every gene in the insect's genome is mutated by a piggyBac transposon or an XP transposon in at least one Drosophila line.


SEQUENCE LISTING (2 sequences)

SEQ ID NO: 1 - DNA, 2480 bp, Trichoplusia ni
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatctcaaagaggtccgacgcgtatgtgccgcaatatatatgacccacttttatgcttcaaactattttttactgatgagataatttcggaaattgtaaaatggacaaatgctgagatatcattgaaacgtcgggaatctatgacaggtgctacatttcgtgacacgaatgaagatgaaatctatgctttctttggtattctggtaatgacagcagtgagaaaagataaccacatgtccacagatgacctctttgatcgatctttgtcaatggtgtacgtctctgtaatgagtcgtgatcgttttgattttttgatacgatgtcttagaatggatgacaaaagtatacggcccacacttcgagaaaacgatgtatttactcctgttagaaaaatatgggatctctttatccatcagtgcatacaaaattacactccaggggctcatttgaccatagatgaacagttacttggttttagaggacggtgtccgtttaggatgtatatcccaaacaagccaagtaagtatggaataaaaatcctcatgatgtgtgacagtggtacgaagtatatgataaatggaatgccttatttgggaagaggaacacagaccaacggagtaccactcggtgaatactacgtgaaggagttatcaaagcctgtgcacggtagttgtcgtaatattacgtgtgacaattggttcacctcaatccctttggcaaaaaacttactacaagaaccgtataagttaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa

SEQ ID NO: 2 - DNA, 10064 bp, mixed species, synthetic
catgatgaaataacataaggtggtcccgtcgatagccgaagcttaccgaagtatacacttaaattcagtgcacgtttgcttgttgagaggaaaggttgtgtgcggacgaatttttttttgaaaacattaacccttacgtggaataaaaaaaaatgaaatattgcaaattttgctgcaaagctgtgactggagtaaaattaattcacgtgccgaagtgtgctattaagagaaaattgtgggagcagagccttgggtgcagccttggtgaaaactcccaaatttgtgatacccactttaatgattcgcagtggaaggctgcacctgcaaaaggtcagacatttaaaaggaggcgactcaacgcagatgccgtacctagtaaagtgatagagcctgaaccagaaaagataaaagaaggctataccagtgggagtacacaaacagagtaagtttgaatagtaaaaaaaatcatttatgtaaacaataacgtgactgtgcgttaggtcctcccgggctgcagatcttgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctagatctcgacgtgttcactttgcttgtttgaattgaattgtcgctccgtagacgaagcgcctctatttatactccggcgctctctcggtaccctcgacccttgccggaggacagtactccgccagcccggaggacagtactccgccttgcgtcggaggacagtactccgacctctagtgtcggaggacagtactccgaccttgcgtcggaggacagtactccgacctctagtgtcggaggacagtactccgaccttgcgtcggaggacagtactccgacctctagtgtcggaggacagtactccgaccttgccggaggacagtactccgcagcccggaggacagtactccgccttgcgtcggaggacagtactccgacctctagtgtcggaggacagtactccgaccttgccggaggacagtactccgccagcccggaggacagtactccgccttggaggcctaagcttgatgacctcgagaaagagagagagaagagaagagagagaacattcgagaaagagagagagaagagaagagagagaacattcgagggggggctagagcggccgcccaattcgcccttgaagatcttgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctagatcttcaagggcgaattgaggtcctgttcattgtttaatgaaaataagagcttgagggaaaaaattcgtactttggagtacgaaatgcgtcgtttagagcagcagccgaattaattctagttccagtgaaatccaagcattttctaaattaaatgtattcttattattatagttgttatttttgatatatataaacaacactattatgcccaccatttttttgagatgcatctacacaaggaacaaacactggatgtcactttcagttcaaattgtaacgctaatcactccgaacaggtcacaaaaaattaccttaaaaagtcataatattaaattagaataaatatagctgtgagggaaatatatacaaatatattggagcaaataaattgtacatacaaatatttattactaatttctattgagacgaaatgaaccactcggaaccatttgagcgaaccgaatcgcgcggaactaacgacagtcgctccaaggtcgtcgaacaaaaggtgaatgtgttgcggagagcgggtgggagacagcgaaagagcaactacgaaacgtggtgtggtggaggtgaattatgaagagggcgcgcgatttgaaaagtatgtatataaaaaatatatcccggtgttttatgtagcgataaacgagtttttgatgtaaggtatgcaggtgtgtaagtcttttggttagaagacaaatccaaagtctacttgtggggatgttcgaaggggaaatacttgtattctataggtcatatcttgtttttattggcacaaatataattacattagctttttgagggggcaataaacagtaaacacgatggtaataatggtaaaaaaaaaaacaagcagttatttcggatatatgtcggctactccttgcgtcgggcccgaagtcttagagccagatatgcgagcacccggaagctcacgatgagaatggccagacccacgtagtccagcggcagatcggcggcggagaagttaagcgtctccaggatgaccttgcccgaactggggcacgtggtgttcgacgatgtgcagctaatttcgcccggctccacgtccgcccattggttaatcagcagaccctcgttggcgtaacggaaccatgagaggtacgacaaccatttgaggtatactggcaccgagcccgagttcaagaagaagccgccaaagagcaggaatggtatgataaccggcggacccacagacagcgccatcgaggtcgaggagctggcgcaggatattagatatccgaaggacgttgacacattggccaccagagtgaccagcgccaggcagttgaagaagtgcagcactccggcccgcagtccgatcatcggataggcaatcgccgtgaagaccagtggcactgtgagaaaaagcggtaattcggcaatcgttttgcccagaaagtatgtgtcacagcgataaagtcgacttcgggcctccctcataaaaactggcagctctgaggtgaacacctaaatcgaatcgattcattagaaagttagtaaattattaatatgcaaatgtattctaaacaagacttacatttatcgtggcaaagacgttttgaaaggtcatgttggtcaggaagaggaagatggctccgttgatattcatcacgcccacttgcgtgagttgttggcccaaaaagatgaggccaatcaagatggcaaccatctgcaaattaaaatgttactcgcatctcattaatattcatatcttcaacatgttcgcgagttaaatgaaatttatttattttctgcaaaactataaactatacatctcattgaaaaaaactaagaagggtgtggaatcaggcaattctaactaaaatctagcgaatttgtttccaagaattgtaagcgttatatcatttgtttccactggaaccactcaccgttgtctgaataagtcgcacttttacgaggagtggttccttgagcaccgacagccaggatcgccacaggaccgcccggaactgcatgaaccaggtggccttgtaggtgtacccattctccggctgctccagtggcttctccaaatttttggtggccaacaactgctccatatcccgggctactttgctaatagcaaaattgtcgcatatcttggcgatccgatcacgggactcgatctcccgtccgggcacaacggccaacacctgtacgtaaaagtccgccggattgtagttggtaggacactgggcacccacgctggataggagttgagatgtaatgtaatgctagatacccttaataaacacatcgaactcactaggaaaagaagtcgacggcttcgctgggagtgcccaagaaagctaccctgccctcggccatcagaaggatcttgtcaaagagctcaaacagctcggaagacggctgatgaatggtcaggatgacggtcttgcccttctgcgacagcttcttcagcacctggacgacgctgtgggcggtaaatgagtccagtccggaggtgggctcatcgcagatcagaagcggcggatcggttagtgcctcggaggcgaatgccagacgcttcctttctccgccggacagacctttcaccctgccgggcacaccgatgatcgtgtgctgacatttgctgagcgaaagctcctggatcacctgatccacgcgggccactcgctgccgataggtcagatgtcgtggcatccgcaccatggcctggaaaatcaggtgttccctggccgttagggagccgataaagaggtcatcctgctggacataggcgcacctggcctgcatctccttggcgtccacaggttggccattgagcagtcgcatcccggatggcgatacttggatgccctgcggcgatcgaaaggcaagggcattcagcagggtcgtctttccggcaccggaactgcccatcacggccaaaagttcgcccggataggccacgccgcaaactgagtttcaaattggtaattggaccctttattaagatttcacacagatcagccgactgcgaatagaaactcaccgttcttgagcaaatgtttcctgggcgccggtatgtgtcgctcgttgcagaatagtccgcgtgtccggttgaccagctgccgccatccggagcccggctgattgaccgccccaaagatgtccatattgtgccaggcataggtgaggttctcggctagttggccgctccctgaaccggagtcctccggcggactgggtggcaggagcgtgccgtagtttttggcctgcccgaagccctggttaatgcagctctgcgaagccgctccgctgtcaccctgcaatgataggggatctcaaatatcaactacaagcgttatgctcatctaaccccgaacaaaacgaagtatcctacgaagtaggtttatacttttatttattttttgtgcatctaggatcagcttaaaatatctggttgttatattttttgtaaaaaagaatgtagtcgaaaatgaatgcctttagatgtcttgatcatgatatgatcttaaaaattgtcttatatagcgagcacagctaccagaataatctgtttcgtgtcactatttgtttgtgcgattgcggtttgggatttttgtgggtcgcagttctcacgccgcagacaatttgatgttgcaatcgcagttcctatagatcaagtgaacttaagatgtatgcacatgtactactcacattgttcagatgctcggcagatgggtgtttgctgcctccgcgaattaatagctcctgatcctcttggcccattgccgggatttttcacactttcccctgcttacccacccaaaaccaatcaccaccccaatcactcaaaaaacaaacaaaaataagaagcgagaggagttttggcacagcactttgtgtttaattgatggcgtaaaccgcttggagcttcgtcacgaaaccgctgacaaagtgcaactgaaggcggacattgacgctaggtaacgctacaaacggtggcgaaagagatagcggacgcagcggcgaaagagacggcgatatttctgtggacagagaaggaggcaaacagcgctgactttgagtggaatgtcattttgagtgagaggtaatcgaaagaacctggtacttcaaatacccttggatcgaagtaaatttaaaactgatcagataagttcaatgatgtccagtgcagtaaaaaataaaaaaaaaatatgtttttttaaatctacattctccaaaaaagggttttattaacttacatacatactagaattcggcttgggtcgaccgctaggtaacgctacaaacggtggcgaaagagatagcggacgcagcggcgaaagagacggcgatatttctgtggacagagaaggaggcaaacagcgctgactttgagtggaatgtcattttgagtgagaggtaatcgaaagaacctggtacatcaaatacccttggatcgaagtaaatttaaaactgatcagataagttcaatgatatccagtgcagtaaaaaaaaaaaatgttttttttatctactttccgcaaaaatgggttttattaacttacatacatactagaattaattcctcgactatggcaagaaaaagtatgcaatataataatcttttattgggtatgcaacgaaaatttgtttcgtcaaagtatgcaatattttttattaaaagagggtatgcaatgtattttattaaaaacgggtatgcaatacaataatcttttattgggtastgcaacgaaaatttgtttcgtcaaagtatgcaatattttttattaaaagagggtatgcaatgtattttattaaaaacgggtatgcaataaaaaattatttggtttctctaaaaagtatgcagcacttattttttgataaggtatgcgacaaaattttactttgccgaaaatatgcaatgtttttgcgaataaattgtcgagcccagtaagccgaattcggtacccgcccgggatcagatccgcggccgctctagccccccctcgaatgttctctctcttctcttctctctctctttctcgaatgttctctctcttctcttctctctctctttctcgaggtcatcaagcttaggcctccaaggcggagtactgtcctccgggctggcggagtactgtcctccggcaaggtcggagtactgtcctccgacactagaggtcggagtactgtcctccgacgcaaggcggagtactgtcctccgggctgcggagtactgtcctccggcaaggtcggagtactgtcctccgacactagaggtcggagtactgtcctccgacgcaaggtcggagtactgtcctccgacactagaggtcggagtactgtcctccgacgcaaggtcggagtactgtcctccgacactagaggtcggagtactgtcctccgacgcaaggcggagtactgtcctccgggctggcggagtactgtcctccggcaagggtcgagggtaccgagagagcgccggagtataaatagaggcgcttcgtctacggagcgacaattcaattcaaacaagcaaagtgaacacgtcgagatctctgcagccaagctttgcgtactcgcaaattattaaaaataaaactttaaaaataatttcgtctaattaatattatgagttaattcaaaccccacggacatgctaagggttaatcaacaatcatatcgctgtctcactcagactcaatacgacactcagaatactattcctttcactcgcacttattgcaagcatacgttaagtggatgtctcttgccgacgggaccaccttatgttatttcatcatggtctggccattctcatcgtgagcttccgggtgctcgcatatctggctctaagacttcgggcccgacgcaaggagtagccgacatatatccgaaataactgcttgtttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggcc